(19) United States
(12) Patent Application Publication
(10) Pub. No.: US 2015/0025449 A1
(43) Pub. Date: Jan. 22, 2015

(54) Activating Peripheral Devices in a Dialysis System

(71) Applicant: Fresenius Medical Care Holdings, Inc., Waltham, MA (US)

(72) Inventors: David Yuds, Antioch, CA (US); Carlos E. Medina, Concord, CA (US)

(73) Assignee: Fresenius Medical Care Holdings, Inc., Waltham, MA (US)

(21) Appl. No.: 13/947,351

(22) Filed: Jul. 22, 2013

(51) Int. Cl. A61M 1/28

(52) U.S. Cl. CPC A61M 1/28
USPC 604/28; 604/29

(57) ABSTRACT

In certain aspects, a method includes determining that one or more alarm criteria of a first alarm condition of a dialysis machine is satisfied, and in response to determining that the one or more alarm criteria of the first alarm condition is satisfied, activating an alarm corresponding to the first alarm condition and activating a peripheral device.

### TECHNICAL FIELD

This disclosure relates to activating peripheral devices in a dialysis system.

### BACKGROUND

Dialysis is a treatment used to support a patient with insufficient renal function. The two principal dialysis methods are hemodialysis and peritoneal dialysis. During hemodialysis (“HD”), the patient's blood is passed through a dialyzer of a dialysis machine while also passing a dialysis solution or dialysate through the dialyzer. A semi-permeable membrane in the dialyzer separates the blood from the dialysate within the dialyzer and allows diffusion and osmosis exchanges to take place between the dialysate and the blood stream. These exchanges across the membrane result in the removal of waste products, including solutes like urea and creatinine, from the blood. These exchanges also regulate the levels of other substances, such as sodium and water, in the blood. In this way, the dialysis machine acts as an artificial kidney for cleansing the blood.

During peritoneal dialysis (“PD”), the patient's peritoneal cavity is periodically infused with dialysate. The membranous lining of the patient's peritoneum acts as a natural semi-permeable membrane that allows diffusion and osmosis exchanges to take place between the solution and the blood stream. These exchanges across the patient's peritoneum result in the removal of waste products, including solutes like urea and creatinine, from the blood, and regulate the levels of other substances, such as sodium and water, in the blood.

Many PD cyclers are designed to automatically infuse, dwell, and drain dialysate to and from the patient's peritoneal cavity. The treatment typically lasts for several hours, often beginning with an initial drain cycle to empty the peritoneal cavity of used or spent dialysate. The sequence then proceeds through the succession of fill, dwell, and drain phases that follow one after the other. Each phase is called a cycle.

### SUMMARY

In one aspect, a method includes determining that one or more alarm criteria of a first alarm condition of a dialysis machine is satisfied, and in response to determining that the one or more alarm criteria of the first alarm condition is satisfied, activating an alarm corresponding to the first alarm condition and activating a peripheral device.

In another aspect, a computer readable storage device stores instructions that, when executed, cause a computer system to perform operations including: (1) accessing a look-up table of a plurality of alarm conditions associated with a dialysis machine, each of the alarm conditions being associated with one or more alarm criteria and one or more peripheral activation data; (2) monitoring one or more states of the dialysis machine, each state being associated with the one or more alarm criteria of at least one of the alarm conditions; (3) in response to determining that the one or more alarm criteria associated with a first alarm condition is satisfied, activating an alarm corresponding to the first alarm condition; and (4) transmitting, to a peripheral device, peripheral activation data associated with the first alarm condition.

In a further aspect, a dialysis system includes a monitoring circuit configured to identify an alarm condition of the dialysis machine. The alarm condition is identified based on one or more criteria associated with the alarm condition, and the alarm condition is associated with peripheral activation data. The dialysis system also includes an alarm circuit configured to transmit the peripheral activation data to a peripheral device based on the identified alarm condition. The peripheral activation data includes data to cause the peripheral device to operate in a mode of the peripheral device relevant to the alarm condition.

Implementations can include one or more of the following features.

In some implementations, the peripheral device is activated by transmitting to the peripheral device peripheral activation data associated with the first alarm condition.

In certain implementations, the peripheral activation data associated with the first alarm condition is wirelessly transmitted to the peripheral device.

In some implementations, determining that the one or more alarm criteria of the first alarm condition is satisfied includes monitoring data associated with the dialysis machine and comparing the data to the one or more alarm criteria of the first alarm condition.

In certain implementations, the data associated with the dialysis machine includes pressure data.

In some implementations, determining that the one or more alarm criteria of the first alarm condition is satisfied includes accessing a look-up table that includes the first alarm condition and the one or more alarm criteria and peripheral activation data associated with the first alarm condition.

In certain implementations, the look-up table includes a plurality of alarm conditions and alarm criteria and peripheral activation data associated with each alarm condition.

In some implementations, the method includes activating a plurality of peripheral devices in response to determining that the one or more alarm criteria of the first alarm condition is satisfied, at least one of the peripheral devices waking a patient upon being activated and at least one of the peripheral devices drawing the patient's attention to a particular part of the system upon being activated.

In certain implementations, the at least one peripheral device that wakes the patient includes a speaker and/or a light directed to the patient, and the at least one peripheral device that draws the patient's attention to a particular part of the system includes a light directed to the particular part of the system.

In some implementations, activating the peripheral device causes the peripheral device to transmit data to a remote location.

In certain implementations, the peripheral includes a mobile phone, and the remote location is a dialysis machine service center.

In some implementations, the method further includes, in response to determining that one or more alarm criteria of a second alarm condition is satisfied, activating an alarm corresponding to the second alarm condition and activating a peripheral device.

In certain implementations, the peripheral device activated in response to determining that the one or more alarm criteria of the second alarm condition is satisfied differs from the peripheral device activated in response to determining that the one or more alarm criteria of the first alarm condition is satisfied.

In some implementations, activating the peripheral device in response to determining that the one or more alarm criteria of the first alarm condition is satisfied causes a first part of the dialysis machine to be illuminated, and activating the peripheral device in response to determining that the one or more alarm criteria of the second alarm condition is satisfied causes a second part of the dialysis machine to be illuminated.

In certain implementations, in response to determining that the one or more alarm criteria of the first alarm condition is satisfied, a plurality of first peripheral devices are activated, at least one of the first peripheral devices waking a patient upon being activated and at least one of the first peripheral devices drawing the patient's attention to a particular part of the system upon being activated.

In some implementations, in response to determining that the one or more alarm criteria of the second alarm condition is satisfied, a plurality of second peripheral devices are activated, at least one of the second peripheral devices waking a patient upon being activated, at least one of the second peripheral devices drawing the patient's attention to a particular part of the system upon being activated, and at least one of the second peripheral devices transmitting data to a remote location.

In certain implementations, at least one of the first peripheral devices and at least one of the second peripheral devices are the same peripheral device.

In some implementations, the peripheral device is electrically isolated from the dialysis machine.

In certain implementations, the first alarm condition is indicative of a condition of a disposable component of the dialysis system.

In some implementations, the first alarm condition is indicative of a malfunction of the disposable component.

In certain implementations, the method includes displaying instructions for remedying the malfunction.

In some implementations, activating the peripheral device causes the peripheral device to provide feedback drawing a patient's attention to the disposable component.

In certain implementations, the feedback includes illuminating the disposable component.

In some implementations, the alarm condition is an indication that a component of the dialysis system may be malfunctioning, and the mode of the peripheral device includes providing feedback drawing attention to the component of the dialysis system.

In certain implementations, providing feedback drawing attention to the component of the dialysis system includes illuminating the component of the dialysis system.

In some implementations, the dialysis system further includes a plurality of peripheral devices that are configured to receive peripheral activation data from the alarm circuit.

In certain implementations, at least one of the peripheral devices is configured to wake a patient upon receiving the peripheral activation data and at least one of the peripheral devices is configured to draw the patient's attention to a particular part of the system upon receiving the peripheral activation data.

In some implementations, the at least one peripheral device that is configured to wake the patient includes a speaker and/or a light directed to the patient, and the at least one peripheral device that is configured to draw the patient's attention to a particular part of the system includes a light directed to the particular part of the system.

In certain implementations, at least one of the peripheral devices is configured to transmit information to a remote location upon receiving the peripheral activation data.

In some implementations, the at least one the peripheral device that is configured to transmit information to the remote location includes a mobile phone, and the remote location is a dialysis machine service center.

In certain implementations, one of the peripheral devices is a light directed to one part of the system, and another of the peripheral devices is a light directed to another part of the system.

In some implementations, the peripheral device is electrically isolated from the dialysis machine.

In certain implementations, the peripheral device is in wireless communication with the dialysis machine.

In some implementations, the peripheral device is configured to illuminate a face of the dialysis machine in response to receiving the peripheral activation data from the alarm circuit.

In certain implementations, the alarm condition is indicative of a condition of a disposable component of the dialysis system.

In some implementations, the alarm condition is indicative of a malfunction of the disposable component.

In certain implementations, the dialysis machine is configured to display instructions for remedying the malfunction.

In some implementations, the mode of the peripheral device includes providing feedback drawing attention to the disposable component.

In certain implementations, the feedback includes illuminating the disposable component.

In some implementations, the disposable component includes a tube.

In certain implementations, the one or more criteria associated with the alarm condition includes a pressure that exceeds a threshold pressure.

In some implementations, the condition of the disposable component includes a kink in the tube.

In certain implementations, the dialysis system includes a look up table of alarm condition entries, each alarm condition entry being associated with i) one or more criteria and ii) peripheral activation data.

In some implementations, the dialysis system includes a user interface, wherein the dialysis machine is configured to display, on the user interface, text related to the alarm condition.

Implementations can include one or more of the following advantages.

In certain implementations, peripheral devices are activated in response to an alarm condition to wake up a patient. The peripheral devices can be especially effective at waking up a patient. For example, the peripheral devices can be selected for or tailored to waking up patients with different needs. In certain implementations, for example, a non-audible peripheral device (e.g., a strobe light) can be used for waking a hearing-impaired patient. In some implementations, an audible peripheral device (e.g., a speaker) can be used for waking a vision-impaired patient.

In certain implementations, peripheral devices can be used to draw the patient's attention to a possible cause of the alarm after the patient has been awoken. In some cases, for example, the part of the dialysis machine likely to have caused the alarm or likely to be affected by the alarm condition can be illuminated so that the patient's attention is drawn to it. By directing the patient's attention to the source of the alarm, the underlying problem can be resolved quickly.

In some implementation, peripheral devices are controlled based on the particular active alarm condition. Different peripheral devices or different combinations of peripheral devices can be activated for different alarms. As a result, those peripheral devices that are best suited to allow the patient to adequately address the particular alarm can be activated in response to the particular alarm. For example, if a malfunction of one part of the dialysis system is likely to cause a first type of alarm and a malfunction of another part of the dialysis system is likely to cause a second type of alarm, then peripheral devices configured to drawn the patient's attention to those respective parts of the dialysis system can be activated in response to the first and second types of alarms, respectively. This can decrease the time required to address an alarm condition. Similarly, the peripheral devices can be activated differently depending on which cycle (i.e., setup, priming, or treatment) is active at the time the alarm occurs. In some implementations, in response to alarms triggered during treatment (which often occurs while the patient is sleeping), peripheral devices configured to wake the patient up and peripheral devices configured to draw the patient's attention to a part of the dialysis system likely to have caused or to have been affected by the alarm condition are activated, and in response to alarms triggered during set up or priming (which typically occur while the patient is awake), only peripheral devices configured to draw the patient's attention to a part of the dialysis system likely to have caused or to have been affected by the alarm condition are activated. Such an arrangement can improve the patient's overall experience by limiting the activation of unnecessary peripheral devices.

In certain implementations, the dialysis machine is configured or programmed to activate peripheral devices in a manner that best addresses a particular patient's needs. For examples, if a patient is hearing-impaired, the dialysis machine can be configured to activate peripherals devices that provide visual feedback or touch feedback rather than audible feedback. Similarly, if a patient is visually-impaired, the dialysis machine can be configured to activate peripherals devices that provide audible-feedback rather than visual feedback. The peripheral devices that are activated in response to various alarm conditions can also be customized according to the patient's preference in certain cases.

In some implementations, peripheral devices communicate wirelessly with the dialysis machine, allowing the peripheral devices to be electrically isolated from the rest of the dialysis machine and reducing the clutter that might be caused by using wires to connect the various different peripheral devices to the dialysis machine.

Other aspects, features, and advantages of the subject matter of this disclosure will be apparent from the description and drawings, and from the claims.

### DESCRIPTION OF DRAWINGS

FIG. 1 is a perspective view of a peritoneal dialysis (“PD”) system that includes a PD cycler positioned atop a portable cart and peripheral devices wirelessly connected to the PD cycler.

FIG. 2 is a perspective view of the PD cycler and a PD cassette of the PD system of FIG. 1, with a door of the PD cycler in the open position to show the inner surfaces of the PD cycler that interface with the PD cassette during use.

FIG. 3 is a perspective view of an open cassette compartment of the PD cycler of FIG. 1, showing, among other things, pistons having piston heads that include spring loaded latch mechanisms that can be used to mechanically connect the piston heads to associated dome-shaped members of the PD cassette.

FIG. 4 is an exploded, perspective view of the PD cassette of FIG. 2, which includes dome-shaped fastening members that can be mechanically connected to the piston heads of the PD cycler of FIG. 1.

FIG. 5 is a perspective, cross-sectional view of the fully assembled PD cassette of FIG. 4.

FIG. 6 is a perspective view of the fully assembled PD cassette of FIG. 4, from a flexible membrane and dome-shaped fastening member side of the PD cassette.

FIG. 7 is a perspective view of the fully assembled PD cassette of FIG. 4, from a rigid base side of the PD cassette.

FIG. 8 is a perspective view of the PD cassette in the cassette compartment of the PD cycler of the PD system of FIG. 1.

FIGS. 9A-9G are diagrammatic cross-sectional views of the PD system of FIG. 1 with the PD cassette disposed in the cassette compartment of the PD cycler, during different phases of a PD treatment and setup.

FIG. 10 is a perspective view of the PD system in which the PD cycler has activated peripheral devices in response to an alarm triggered by a kink in the patient line.

FIG. 11 is a perspective view of the PD system in which the PD cycler has activated peripheral devices in response to an alarm triggered by a leak in the patient line.

FIG. 12 is a perspective view of the PD system in which the PD cycler has activated peripheral devices in response to an alarm triggered by a loss of main power to the PD cycler.

FIG. 13 shows a computer system and related components.

FIG. 14 is a flowchart showing operations carried out by the PD cycler in connection with activating one or more of the peripheral devices.

### DETAILED DESCRIPTION

In some aspects, a PD system includes peripheral devices (e.g., speakers, cellular telephone, and/or lamps) in communication with (e.g., wirelessly connected to) a PD cycler and used in connection with alarms triggered at the PD cycler. For example, one or more of the peripheral devices can be activated in response to an alarm. In certain implementations, different alarms will activate different peripheral devices or different combinations of peripheral devices to direct the patient's attention to different components of the PD system. Activation of the peripheral devices in response to the alarm can get the attention of the patient (e.g., wake up the patient) and direct the patient's attention to components of the system likely to be causing or affected by the alarm condition.

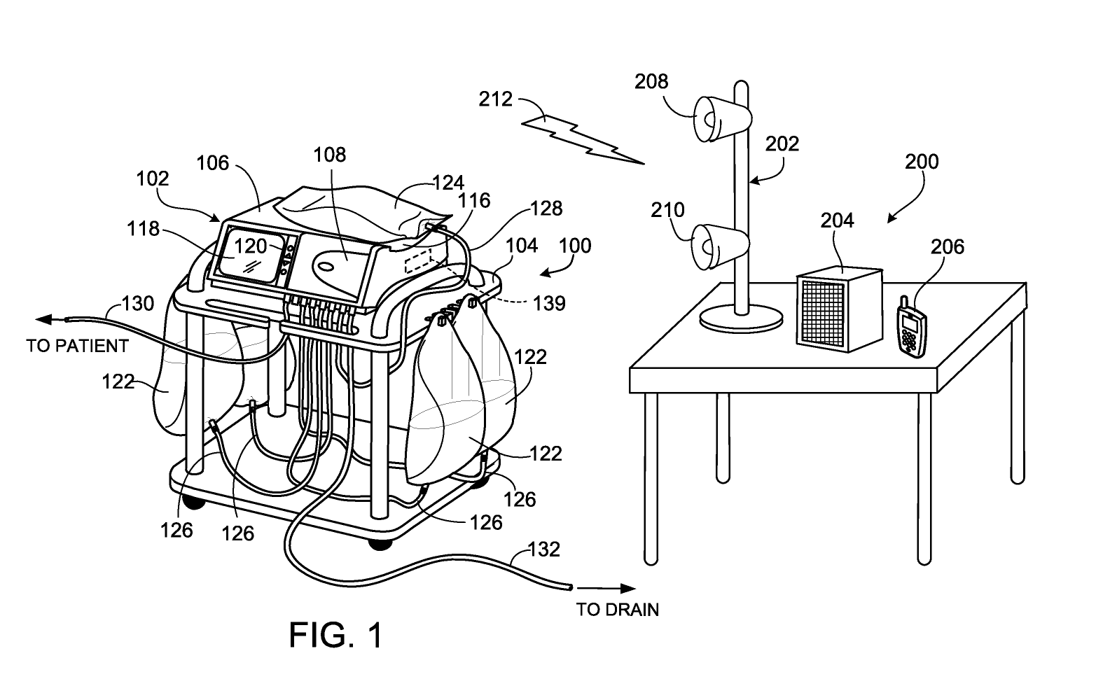

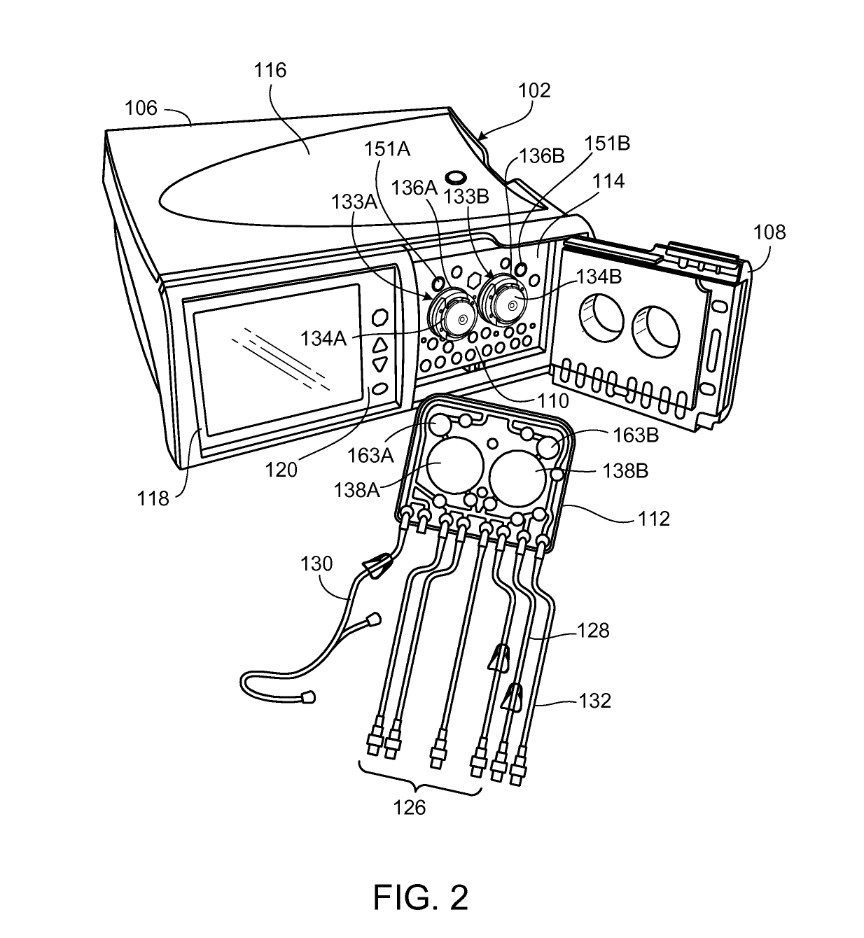

Referring to FIG. 1, a peritoneal dialysis (“PD”) system 100 includes a PD cycler (also referred to as a PD machine) 102 seated on a cart 104. Referring also to FIG. 2, the PD cycler 102 includes a housing 106, a door 108, and a cassette interface 110 that contacts a disposable PD cassette 112 when the cassette 112 is disposed within a cassette compartment 114 formed between the cassette interface 110 and the closed door 108. A heater tray 116 is positioned on top of the housing 106. The heater tray 116 is sized and shaped to accommodate a bag of dialysate (e.g., a 5 liter bag of dialysate). The PD cycler 102 also includes a touch screen 118 and additional control buttons 120 that can be operated by a user (e.g., a patient) to allow, for example, set-up, initiation, and/or termination of a PD treatment.

Dialysate bags 122 are suspended from fingers on the sides of the cart 104, and a heater bag 124 is positioned in the heater tray 116. The dialysate bags 122 and the heater bag 124 are connected to the cassette 112 via dialysate bag lines 126 and a heater bag line 128, respectively. The dialysate bag lines 126 can be used to pass dialysate from dialysate bags 122 to the cassette 112 during use, and the heater bag line 128 can be used to pass dialysate back and forth between the cassette 112 and the heater bag 124 during use. In addition, a patient line 130 and a drain line 132 are connected to the cassette 112. The patient line 130 can be connected to a patient's abdomen via a catheter and can be used to pass dialysate back and forth between the cassette 112 and the patient's peritoneal cavity during use. The drain line 132 can be connected to a drain or drain receptacle and can be used to pass dialysate from the cassette 112 to the drain or drain receptacle during use.

As shown in FIG. 1, the PD system 100 further includes peripheral devices 200 under control of the PD cycler 102. The peripheral devices 200 shown include a light 202, a speaker 204, and a cellular telephone 206. The light 202 includes a lighting element 210 pointed towards the PD cycler 102, and another lighting element 208 pointed towards the patient.

The peripheral devices 200 are typically electrically isolated from the PD cycler 102 so that the electronics of the PD cycler 102 will not be electrically affected by the operation of the peripheral devices 200. For example, the peripheral devices 200 can be in wireless communication with the PD cycler 102. In some implementations, a wireless transceiver of the PD cycler 102 communicates with one or more of the peripheral devices 200 using wireless signals 212. The peripheral devices 200 can, for example, communicate with the PD cycler 102 using standard wireless communication protocols (e.g., 802.11, Bluetooth, X10, etc.). The PD cycler 102, as will be described in greater detail below, can activate one or more of the peripheral devices 200 during use (e.g., in response to an alarm condition) to get the attention of the patient and/or to draw the attention of the patient to a region of the PD system 100 likely to be experiencing a complication.

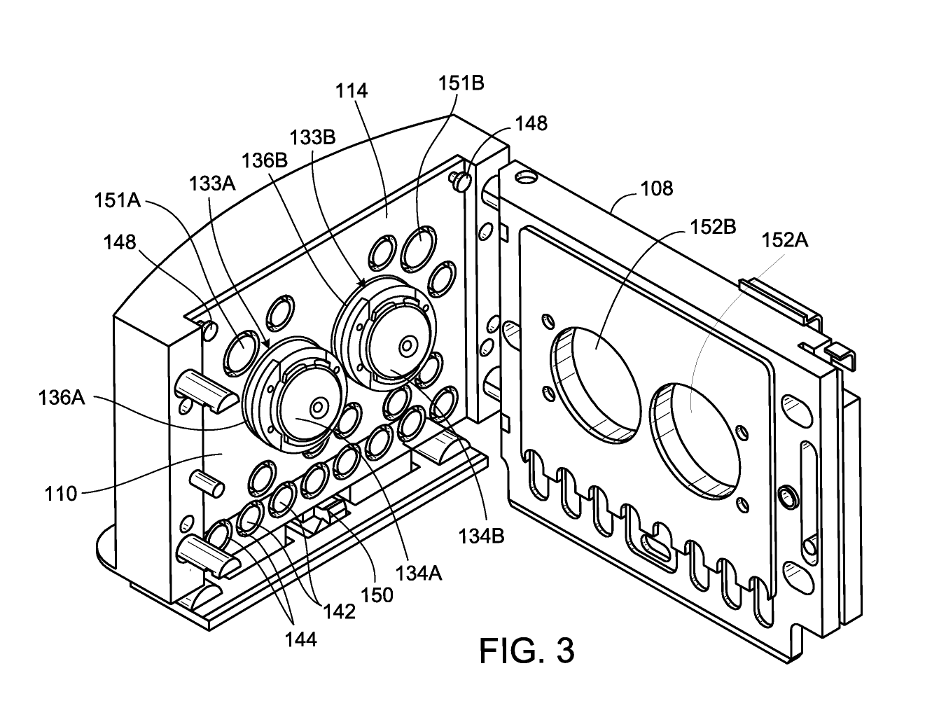

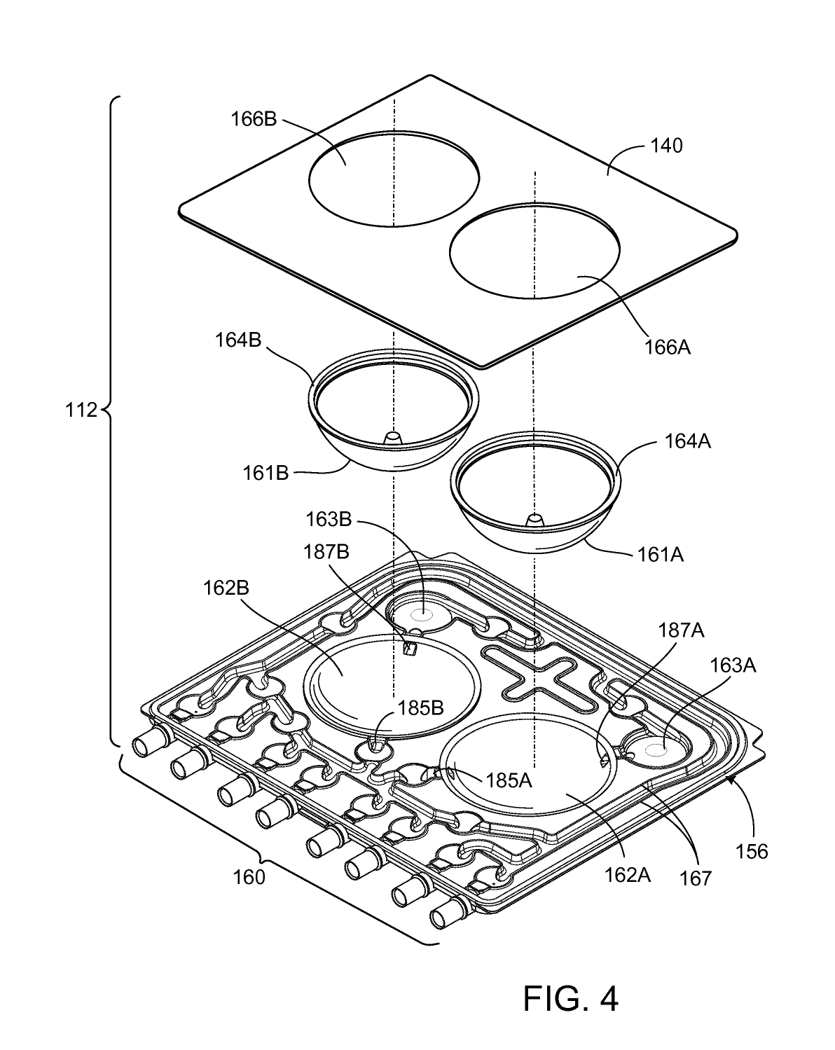

FIG. 3 shows a more detailed view of the cassette interface 110 and the door 108 of the PD cycler 102. As shown, the PD cycler 102 includes pistons 133A, 133B with piston heads 134A, 134B attached to piston shafts 135A, 135B (piston shaft 135A shown in FIG. 4) that can be axially moved within piston access ports 136A, 136B formed in the cassette interface 110. The piston shafts 135A, 135B are connected to stepper motors that can be operated to move the pistons 133A, 133B axially inward and outward such that the piston heads 134A, 134B move axially inward and outward within the piston access ports 136A, 136B. The stepper motors drive lead screws, which move nuts inward and outward along the lead screws. The nuts, in turn, are connected to the pistons 133A, 133B and thus cause the pistons 133A, 133B to move inward and outward as the stepper motors rotate the lead screws. Stepper motor controllers provide the necessary current to be driven through the windings of the stepper motors to move the pistons 133A, 133B. The polarity of the current determines whether the pistons 133A, 133B are advanced or retracted. In some implementations, the stepper motors require 200 steps to make a full rotation, and this corresponds to 0.048 inch of linear travel.

The PD system 100 also includes encoders (e.g., optical encoders) that measure the rotational movement of the lead screws. The axial positions of the pistons 133A, 133B can be determined based on the rotational movement of the lead screws, as determined by the encoders. Thus, the measurements of the encoders can be used to accurately position the piston heads 134A, 134B of the pistons 133A, 133B.

As discussed below, when the cassette 112 (shown in FIGS. 2 and 4-7) is positioned within the cassette compartment 114 of the PD cycler 102 with the door 108 closed, the piston heads 134A, 134B of the PD cycler 102 align with pump chambers 138A, 138B of the cassette 112 such that the piston heads 134A, 134B can be mechanically connected to dome-shaped fastening members 161A, 161B of the cassette 112 overlying the pump chambers 138A, 138B. As a result of this arrangement, movement of the piston heads 134A, 134B toward the cassette 112 during treatment can decrease the volume of the pump chambers 138A, 138B and force dialysate out of the pump chambers 138A, 138B, while retraction of the piston heads 134A, 134B away from the cassette 112 can increase the volume of the pump chambers 138A, 138B and cause dialysate to be drawn into the pump chambers 138A, 138B.

As shown in FIG. 3, the cassette interface 110 includes two pressure sensors 151A, 151B that align with pressure sensing chambers 163A, 163B (shown in FIGS. 2, 4, 6, and 7) of the cassette 112 when the cassette 112 is positioned within the cassette compartment 114. Portions of a membrane 140 of the cassette 112 that overlie the pressure sensing chambers 163A, 163B adhere to the pressure sensors 151A, 151B using vacuum pressure. Specifically, clearance around the pressure sensors 151A, 151B communicates vacuum to the portions of the cassette membrane 140 overlying the pressure sensing chambers 163A, 163B to hold those portions of the cassette membrane 140 tightly against the pressure sensors 151A, 151B. The pressure of fluid within the pressure sensing chambers 163A, 163B causes the portions of the cassette membrane 140 overlying the pressure sensing chambers 163A, 163B to contact and apply pressure to the pressure sensors 151A, 151B.

The pressure sensors 151A, 151B can be any sensors that are capable of sensing the fluid pressure in the sensing chambers 163A, 163B. In some implementations, the pressure sensors are solid state silicon diaphragm infusion pump force/pressure transducers. One example of such a sensor is the Model 1865 force/pressure transducer manufactured by Sensym Foxboro ICT. In certain implementations, the force/pressure transducer is modified to provide increased voltage output. The force/pressure transducer can, for example, be modified to produce an output signal of 0 to 5 volts.

Still referring to FIG. 3, the PD cycler 102 also includes multiple inflatable members 142 positioned within inflatable member ports 144 in the cassette interface 110. The inflatable members 142 align with depressible dome regions 146 of the cassette 112 (shown in FIGS. 4-6) when the cassette 112 is positioned within the cassette compartment 114 of the PD cycler 102. While only a couple of the inflatable members 142 are labeled in FIG. 3, it should be understood that the PD cycler 102 includes an inflatable member 142 associated with each of the depressible dome regions 146 of the cassette 112. The inflatable members 142 act as valves to direct dialysate through the cassette 112 in a desired manner during use. In particular, the inflatable members 142 bulge outward beyond the surface of the cassette interface 110 and into contact with the depressible dome regions 146 of the cassette 112 when inflated, and retract into the inflatable member ports 144 and out of contact with the cassette 112 when deflated. By inflating certain inflatable members 142 to depress their associated dome regions 146 on the cassette 112, certain fluid flow paths within the cassette 112 can be occluded. Thus, dialysate can be pumped through the cassette 112 by actuating the piston heads 134A, 134B, and can be guided along desired flow paths within the cassette 112 by selectively inflating and deflating the various inflatable members 142.

Still referring to FIG. 3, locating pins 148 extend from the cassette interface 110 of the PD cycler 102. When the door 108 is in the open position, the cassette 112 can be loaded onto the cassette interface 110 by positioning the top portion of the cassette 112 under the locating pins 148 and pushing the bottom portion of the cassette 112 toward the cassette interface 110. The cassette 112 is dimensioned to remain securely positioned between the locating pins 148 and a spring loaded latch 150 extending from the cassette interface 110 to allow the door 108 to be closed over the cassette 112. The locating pins 148 help to ensure that proper alignment of the cassette 112 within the cassette compartment 114 is maintained during use.

The door 108 of the PD cycler 102, as shown in FIG. 3, defines cylindrical recesses 152A, 152B that substantially align with the pistons 133A, 133B when the door 108 is in the closed position. When the cassette 112 (shown in FIGS. 4-7) is positioned within the cassette compartment 114, hollow projections 154A, 154B of the cassette 112, inner surfaces of which partially define the pump chambers 138A, 138B, fit within the recesses 152A, 152B. The door 108 further includes a pad that is inflated during use to compress the cassette 112 between the door 108 and the cassette interface 110. With the pad inflated, the portions of the door 108 forming the recesses 152A, 152B support the projections 154A, 154B of the cassette 112 and the planar surface of the door 108 supports the other regions of the cassette 112. The door 108 can counteract the forces applied by the inflatable members 142 and thus allows the inflatable members 142 to actuate the depressible dome regions 146 on the cassette 112. The engagement between the door 108 and the hollow projections 154A, 154B of the cassette 112 can also help to hold the cassette 112 in a desired fixed position within the cassette compartment 114 to further ensure that the pistons 133A, 133B align with the fluid pump chambers 138A, 138B of the cassette 112.

A control unit (e.g., microprocessor) 139 (shown in FIG. 1) is connected to the pressure sensors 151A, 151B, to the stepper motors (e.g., the drivers of the stepper motors) that drive the pistons 133A, 133B, and to the encoders that monitor rotation of the lead screws of the stepper motors such that the control unit 139 can receive signals from and transmit signals to those components of the system. As will be described in greater detail below, the control unit 139 monitors the components to which it is connected to determine whether any complications exists within the PD system 100. In the event of complications, the control unit 139 triggers one or more alarms and initiates communication (e.g., wirelessly) to activate one or more of the peripheral devices 200. The peripheral devices 200 can, for example, be activated in a manner to get the attention of the patient and/or to draw the attention of the patient to a region of the PD system 100 determined to be experiencing the complication. In some implementations, the control unit 139 is an MPC823 PowerPC device manufactured by Motorola, Inc.

The PD cycler 102 has a backup power source (e.g. a battery) connected to the control unit 139. In the event of a power loss, the backup power source enables transmission of peripheral activation with the peripheral devices 200.

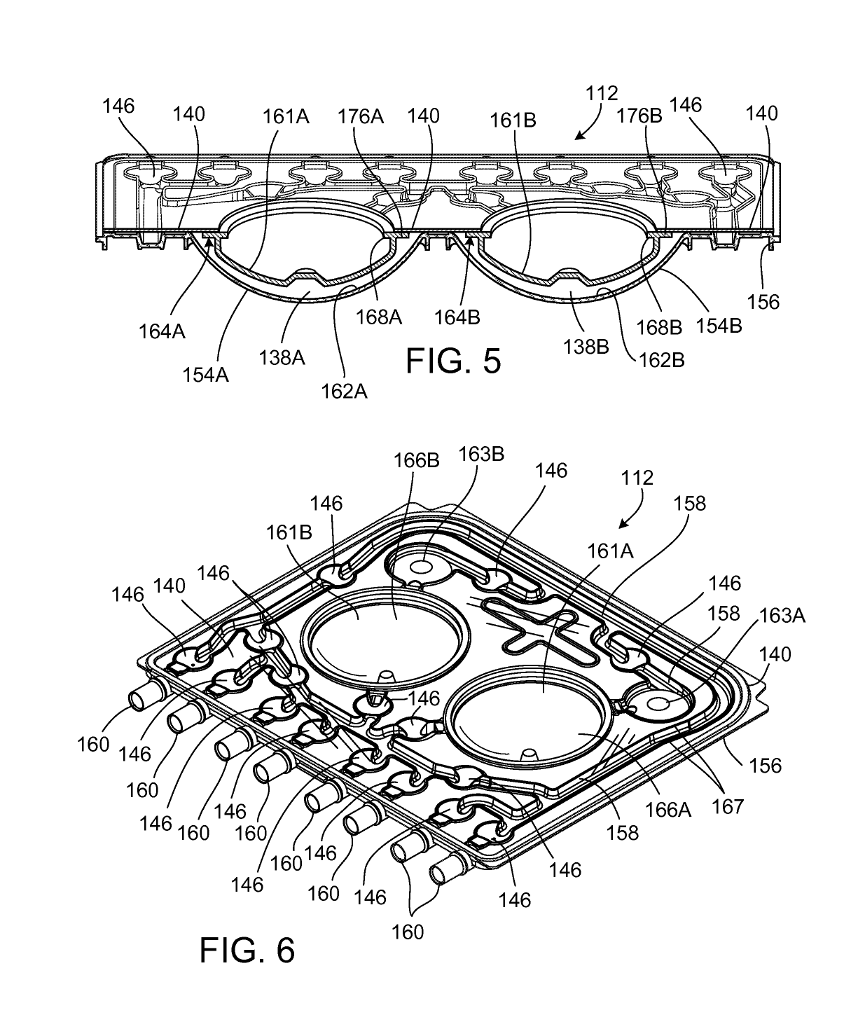

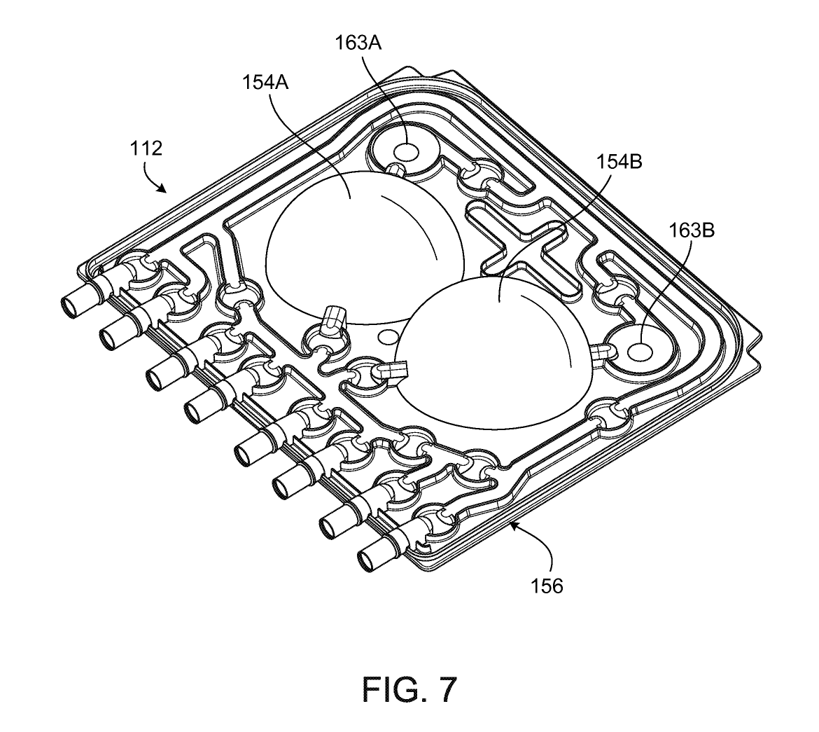

FIG. 4 is an exploded, perspective view of the cassette 112, FIG. 5 is a perspective, cross-sectional view of the fully assembled cassette 112, and FIGS. 6 and 7 are perspective views of the assembled cassette 112, from the membrane side and from the rigid base side, respectively. Referring to FIGS. 4-6, the flexible membrane 140 of the cassette 112 is attached to a periphery of the tray-like rigid base 156. Rigid dome-shaped fastening members 161A, 161B are positioned within recessed regions 162A, 162B of the base 156. The dome-shaped fastening members 161A, 161B are sized and shaped to receive the piston heads 134A, 134B of the PD cycler 102. In certain implementations, the dome-shaped fastening members 161A, 161B have a diameter, measured from the outer edges of flanges 164A, 164B, of about 1.5 inches to about 2.5 inches (e.g., about 2.0 inches) and take up about two-thirds to about three-fourths of the area of the recessed regions 162A, 162B. The annular flanges 164A, 164B of the rigid dome-shaped fastening members 161A, 161B are attached in a liquid-tight manner to portions of the inner surface of the membrane 140 surrounding substantially circular apertures 166A, 166B formed in the membrane 140. The annular flanges 164A, 164B of the rigid dome-shaped fastening members 161A, 161B can, for example, be thermally bonded or adhesively bonded to the membrane 140. The apertures 166A, 166B of the membrane 140 expose the rigid dome-shaped fastening members 161A, 161B such that the piston heads 134A, 134B are able to directly contact and mechanically connect to the dome-shaped fastening members 161A, 161B during use.

The annular flanges 164A, 164B of the dome-shaped fastening members 161A, 161B, as shown in FIG. 5, form annular projections 168A, 168B that extend radially inward and annular projections 176A, 176B that extend radially outward from the side walls of the dome-shaped fastening members 161A, 161B. When the piston heads 134A, 134B are mechanically connected to the dome-shaped fastening members 161A, 161B, the radially inward projections 168A, 168B engage the rear angled surfaces of the sliding latches 145A, 147A of the piston heads 134A, 134B to firmly secure the dome-shaped fastening members 161A, 161B to the piston heads 134A, 134B. Because the membrane 140 is attached to the dome-shaped fastening members 161A, 161B, movement of the dome-shaped fastening members 161A, 161B into and out of the recessed regions 162A, 162B of the base 156 (e.g., due to reciprocating motion of the pistons 133A, 133B) causes the flexible membrane 140 to similarly be moved into and out of the recessed regions 162A, 162B of the base 156. This movement allows fluid to be forced out of and drawn into the fluid pump chambers 138A, 138B, which are formed between the recessed regions 162A, 162B of the base 156 and the portions of the dome-shaped fastening members 161A, 161B and membrane 140 that overlie those recessed regions 162A, 162B.

Referring to FIGS. 4 and 6, raised ridges 167 extend from the substantially planar surface of the base 156 towards and into contact with the inner surface of the flexible membrane 140 when the cassette 112 is compressed between the door 108 and the cassette interface 110 of the PD cycler 102 to form a series of fluid passageways 158 and to form the multiple, depressible dome regions 146, which are widened portions (e.g., substantially circular widened portions) of the fluid pathways 158, as shown in FIG. 6. The fluid passageways 158 fluidly connect the fluid line connectors 160 of the cassette 112, which act as inlet/outlet ports of the cassette 112, to the fluid pump chambers 138A, 138B. As noted above, the various inflatable valve members 142 of the PD cycler 102 act on the cassette 112 during use. During use, the dialysate flows to and from the pump chambers 138A, 138B through the fluid pathways 158 and dome regions 146. At each depressible dome region 146, the membrane 140 can be deflected to contact the planar surface of the base 156 from which the raised ridges 167 extend. Such contact can substantially impede (e.g., prevent) the flow of dialysate along the region of the pathway 158 associated with that dome region 146. Thus, the flow of dialysate through the cassette 112 can be controlled through the selective depression of the depressible dome regions 146 by selectively inflating the inflatable members 142 of the PD cycler 102.

Still referring to FIGS. 4 and 6, the fluid line connectors 160 are positioned along the bottom edge of the cassette 112. As noted above, the fluid pathways 158 in the cassette 112 lead from the pumping chambers 138A, 138B to the various connectors 160. The connectors 160 are positioned asymmetrically along the width of the cassette 112. The asymmetrical positioning of the connectors 160 helps to ensure that the cassette 112 will be properly positioned in the cassette compartment 114 with the membrane 140 of the cassette 112 facing the cassette interface 110. The connectors 160 are configured to receive fittings on the ends of the dialysate bag lines 126, the heater bag line 128, the patient line 130, and the drain line 132. One end of the fitting can be inserted into and bonded to its respective line and the other end can be inserted into and bonded to its associated connector 160. By permitting the dialysate bag lines 126, the heater bag line 128, the patient line 130, and the drain line 132 to be connected to the cassette, as shown in FIGS. 1 and 2, the connectors 160 allow dialysate to flow into and out of the cassette 112 during use. As the pistons 133A, 133B are reciprocated, the inflatable members 142 can be selectively inflated to allow fluid to flow from any of the lines 126, 128, 130, and 132 to any of ports 185A, 185B, 187A, and 187B of the pump chambers 138A, 138B, and vice versa.

The rigidity of the base 156 helps to hold the cassette 112 in place within the cassette compartment 114 of the PD cycler 102 and to prevent the base 156 from flexing and deforming in response to forces applied to the projections 154A, 154B by the dome-shaped fastening members 161A, 161B and in response to forces applied to the planar surface of the base 156 by the inflatable members 142. The dome-shaped fastening members 161A, 161B are also sufficiently rigid that they do not deform as a result of usual pressures that occur in the pump chambers 138A, 138B during the fluid pumping process. Thus, the deformation or bulging of the annular portions 149A, 149B of the membrane 140 can be assumed to be the only factor other than the movement of the pistons 133A, 133B that affects the volume of the pump chambers 138A, 138B during the pumping process.

The base 156 and the dome-shaped fastening members 161A, 161B of the cassette 112 can be formed of any of various relatively rigid materials. In some implementations, these components of the cassette 112 are formed of one or more polymers, such as polypropylene, polyvinyl chloride, polycarbonate, polysulfone, and other medical grade plastic materials. In certain implementations, these components can be formed of one or more metals or alloys, such as stainless steel. These components of can alternatively be formed of various different combinations of the above-noted polymers and metals. These components of the cassette 112 can be formed using any of various different techniques, including machining, molding, and casting techniques.

As noted above, the membrane 140 is attached to the periphery of the base 156 and to the annular flanges 164A, 164B of the dome-shaped fastening members 161A, 161B. The portions of the membrane 140 overlying the remaining portions of the base 156 are typically not attached to the base 156. Rather, these portions of the membrane 140 sit loosely atop the raised ridges 165A, 165B, and 167 extending from the planar surface of the base 156. Any of various attachment techniques, such as adhesive bonding and thermal bonding, can be used to attach the membrane 140 to the periphery of the base 156 and to the dome-shaped fastening members 161A, 161B. The thickness and material(s) of the membrane 140 are selected so that the membrane 140 has sufficient flexibility to flex toward the base 156 in response to the force applied to the membrane 140 by the inflatable members 142. In certain implementations, the membrane 140 is about 0.100 micron to about 0.150 micron in thickness. However, various other thicknesses may be sufficient depending on the type of material used to form the membrane 140.

Any of various different materials that permit the membrane 140 to deflect in response to movement of the inflatable members 142 without tearing can be used to form the membrane 140. In some implementations, the membrane 140 includes a three-layer laminate. In certain implementations, for example, inner and outer layers of the laminate are formed of a compound that is made up of 60 percent Septon® 8004 thermoplastic rubber (i.e., hydrogenated styrenic block copolymer) and 40 percent ethylene, and a middle layer is formed of a compound that is made up of 25 percent Tuftec® H1062 (SEBS: hydrogenated styrenic thermoplastic elastomer), 40 percent Engage® 8003 polyolefin elastomer (ethylene octene copolymer), and 35 percent Septon® 8004 thermoplastic rubber (i.e., hydrogenated styrenic block copolymer). The membrane can alternatively include more or fewer layers and/or can be formed of different materials.

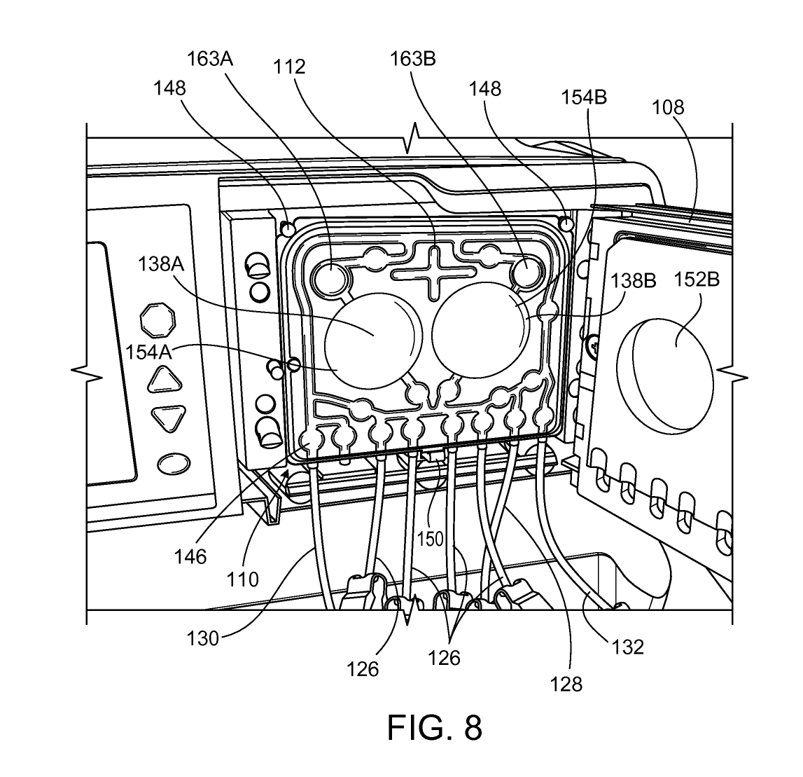

As shown in FIG. 8, before treatment, the door 108 of the PD cycler 102 is opened to expose the cassette interface 110, and the cassette 112 is positioned with its dome-shaped fastening members 161A, 161B aligned with the pistons 133A, 133B of the PD cycler 102, its pressure sensing chambers 163A, 163B aligned with the pressure sensors 151A, 151B of the PD cycler, its depressible dome regions 146 aligned with the inflatable members 142 of the PD cycler 102, and its membrane 140 adjacent to the cassette interface 110. In order to ensure that the cassette 112 is properly positioned on the cassette interface 110, the cassette 112 is positioned between the locating pins 148 and the spring loaded latch 150 extending from the cassette interface 110. The asymmetrically positioned connectors 160 of the cassette act as a keying feature that reduces the likelihood that the cassette 112 will be installed with the membrane 140 and dome-shaped fastening members 161A, 161B facing in the wrong direction (e.g., facing outward toward the door 108). Additionally or alternatively, the locating pins 148 can be dimensioned to be less than the maximum protrusion of the projections 154A, 154B such that the cassette 112 cannot contact the locating pins 148 if the membrane 140 is facing outward toward the door 108. The pistons 133A, 133B are typically retracted into the piston access ports 136A, 136B during installation of the cassette 112 to avoid interference between pistons 133A, 133B and the dome-shaped fastening members 161A, 161B and thus increase the ease with which the cassette 112 can be positioned within the cassette compartment 114.

After positioning the cassette 112 as desired on the cassette interface 110, the door 108 is closed and the inflatable pad within the door 108 is inflated to compress the cassette 112 between the inflatable pad and the cassette interface 110. This compression of the cassette 112 holds the projections 154A, 154B of the cassette 112 in the recesses 152A, 152B of the door 108 and presses the membrane 140 tightly against the raised ridges 167 extending from the planar surface of the rigid base 156 to form the enclosed fluid pathways 158 and dome regions 146 (shown in FIG. 6). Referring briefly also to FIGS. 1 and 2, the patient line 130 is then connected to a patient's abdomen via a catheter, and the drain line 132 is connected to a drain or drain receptacle. In addition, the heater bag line 128 is connected to the heater bag 124, and the dialysate bag lines 126 are connected to the dialysate bags 122. At this point, the pistons 133A, 133B can be coupled to dome-shaped fastening members 161A, 161B of the cassette 112 to permit priming of the cassette 112 and the lines 126, 128, 130, 132. Once these components have been primed, treatment can be initiated.

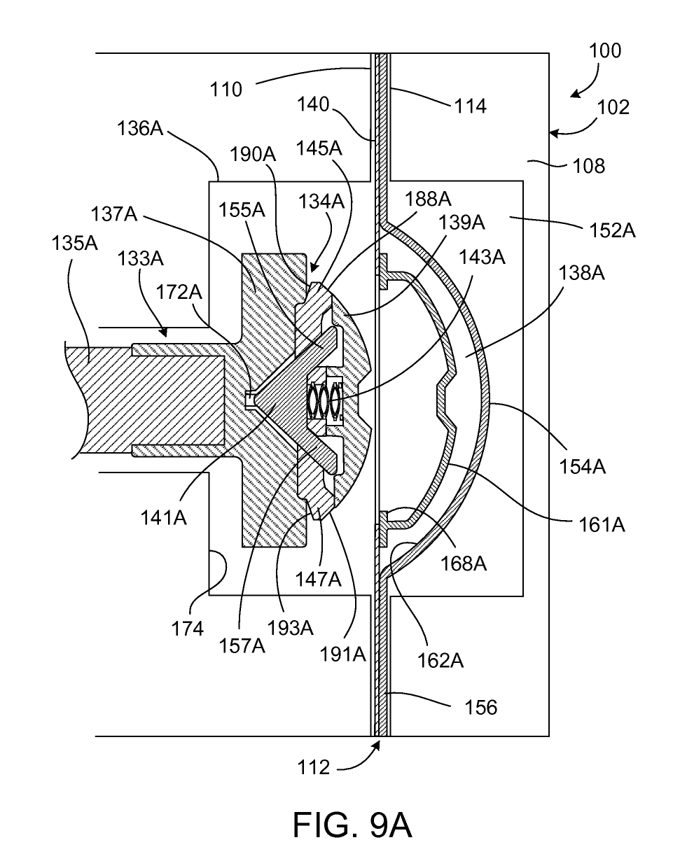

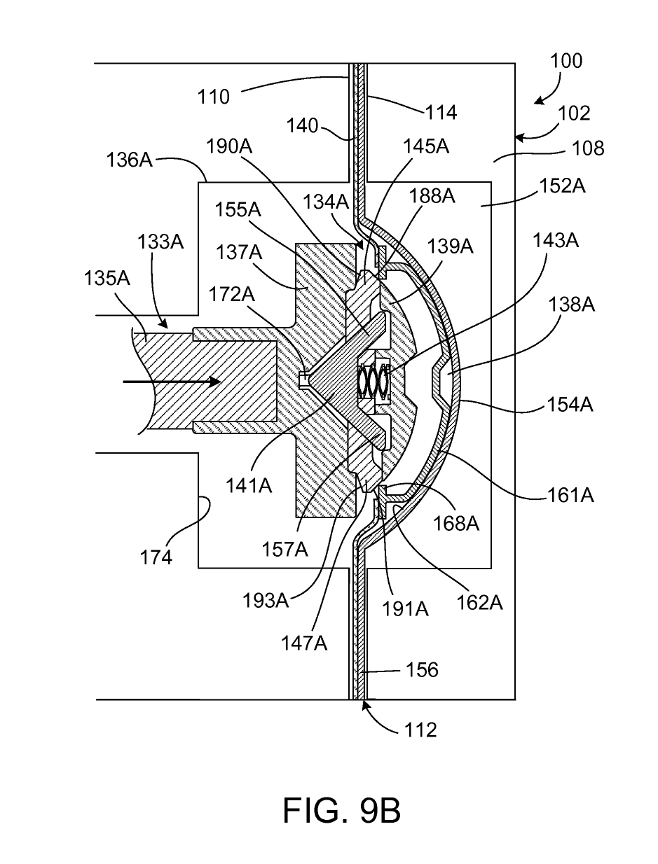

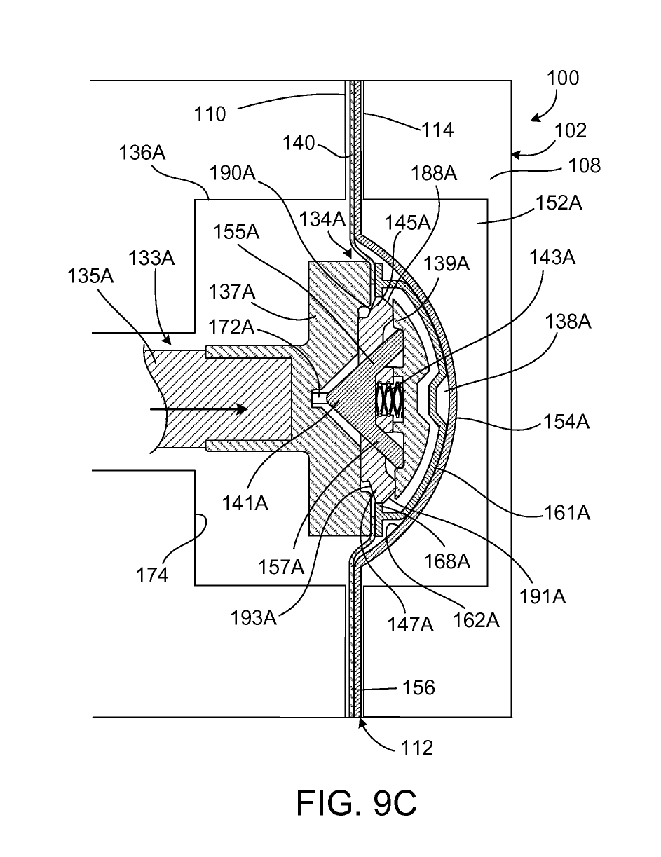

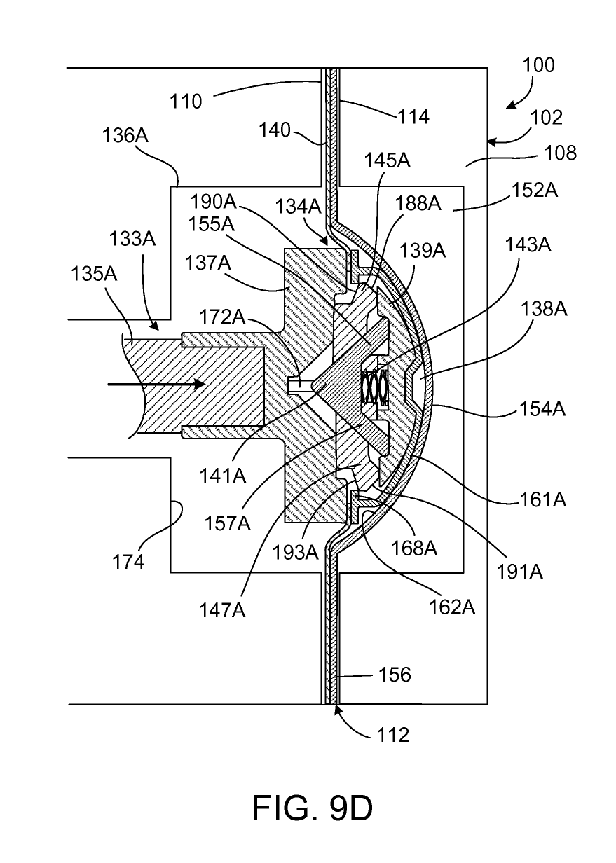

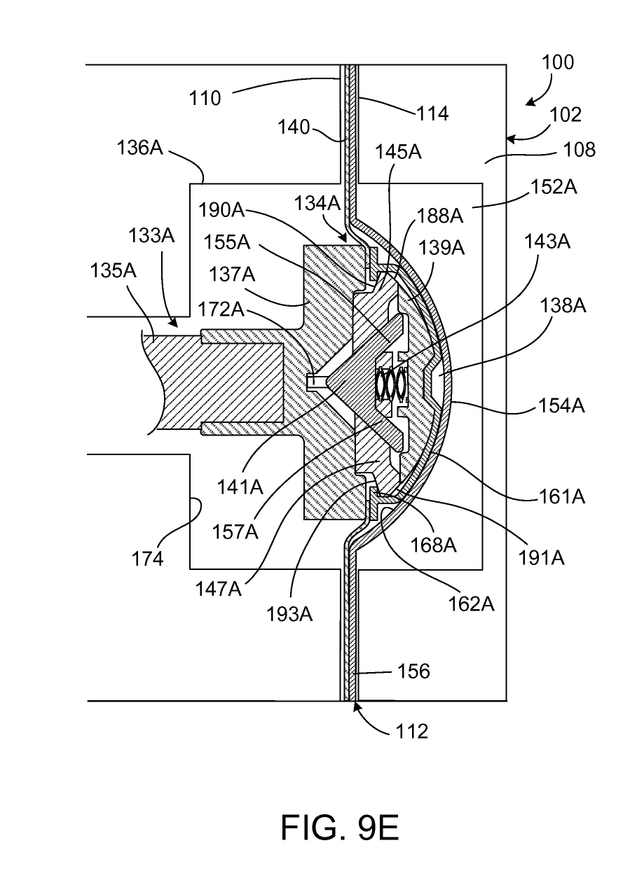

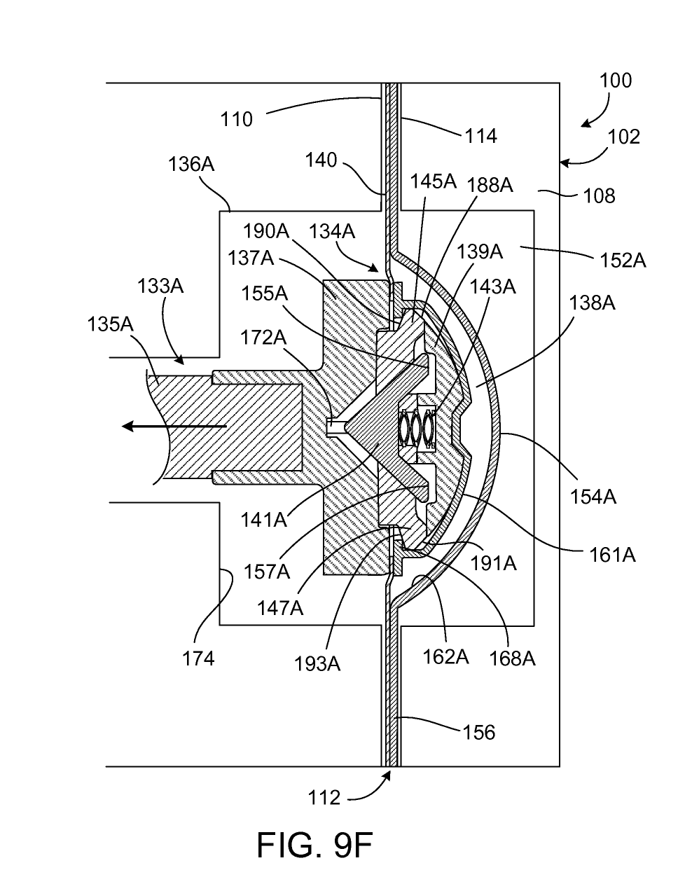

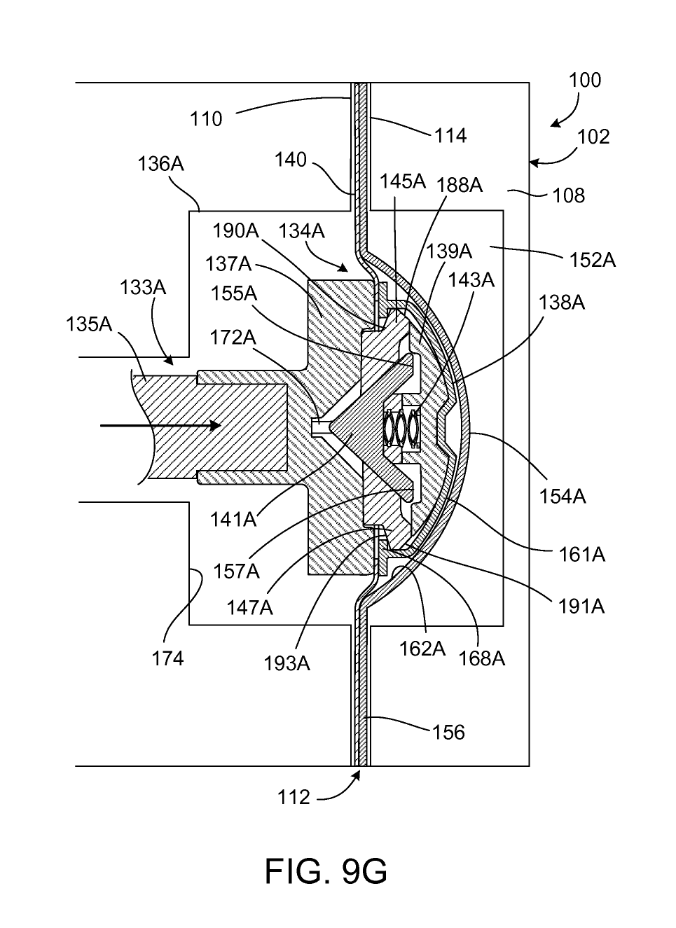

FIGS. 9A-9G, which will be discussed below, are cross-sectional views of the system during different stages of the setup, priming, and treatment. These figures focus on the interaction between the piston 133A of the PD cycler 102 and the pump chamber 138A of the cassette 112 during the setup, priming, and treatment. The interaction between the other piston 133B and pump chamber 138B is identical and thus will not be separately described in detail.

FIG. 9A shows the piston 133A fully retracted into the piston access port 136A of the cassette interface 110. The cassette 112 is positioned in the cassette compartment 114 of the PD cycler 102 and the inflatable pad in the door 108 of the PD cycler 102 is inflated such that the cassette 112 is pressed tightly against the cassette interface 110 of the PD cycler 102, as explained above.

Referring to FIG. 9B, with the cassette 112 properly installed within the cassette compartment 114 of the PD cycler 102 and the appropriate line connections made, the piston 133A is advanced to initiate the process of mechanically connecting the piston head 134A of the PD cycler 102 to the dome-shaped fastening member 161A of the cassette 112. As the piston 133A is advanced, a front angled surface 188A of a sliding latch 145A and a front angled surface 191A of a sliding latch 147A contact a rear surface of the annular projection 168A, which extends radially inward from the dome-shaped fastening member 161A. The rear surface of the annular projection 168A is approximately perpendicular to the longitudinal axis of the piston 133A.

As the piston 133A continues to advance, the dome-shaped fastening member 161A contacts the inner surface of the portion of the rigid base 156 that forms the recessed region 162A, as shown in FIG. 9B. The rigid base 156 prevents further forward movement of the dome-shaped fastening member 161A. The membrane 140, which is attached to the peripheral flange 164A of the dome-shaped fastening member 161A, also stretches and moves into the recessed region 162A due to the advancing piston 133A. Due to the angled geometries of the front angled surfaces 188A, 191A of the sliding latches 145A, 147A and the resistance provided by the rigid base 156 to the forward motion of the dome-shaped fastening member 161A, the sliding latches 145A, 147A are caused to move radially inward (i.e., toward the longitudinal axis of the piston 133A) as the piston head 134A continues to be advanced relative to the dome-shaped fastening member 161A. More specifically, the forward motion of the sliding latches 145A, 147A is converted into a combined forward and radially inward motion due to the sliding motion of the front angled surfaces 188A, 191A of the sliding latches 145A, 147A against the rear surface of the annular projection 168A of the dome-shaped fastening member 161A. The radial inward movement of each of the sliding latches 145A, 147A in turn causes a forward movement of a latch lock 141A of the piston head 134A due to the mated geometries of the outer surfaces of legs 155A, 157A of the latch lock 141A and the surfaces of the sliding latches 145A, 147A that are positioned adjacent to and brought into contact with those outer surfaces of the legs 155A, 157A. This forward movement of the latch lock 141A is resisted by a spring 143A in the piston head. FIG. 9C shows the piston head 134A at a point during the connection process at which the sliding latches 145A, 147A have been deflected radially inward a sufficient distance to allow the sliding latches 145A, 147A to pass beyond the annular projection 168A that extends radially inward from the dome-shaped fastening member 161A. In this position, outer peripheral surfaces of the sliding latches 145A, 147A, which are substantially parallel to the longitudinal axis of the piston 133A, contact and slide along an inner surface of the annular projection 168A of the dome-shaped fastening member 161A, which is also substantially parallel to the longitudinal axis of the piston 133A. The spring 143A is further compressed due to the radially inwardly deflected positions of the sliding latches 145A, 147A.

Referring to FIG. 9D, as the sliding latches 145A, 147A pass beyond the annular projection 168A, the spring 143A is allowed to expand. The expansion of the spring 143A causes the latch lock 141A to move rearward. As a result, the outer surfaces of the legs 155A, 157A of the latch lock 141A contact the correspondingly angled adjacent surfaces of the sliding latches 145A, 147A, causing the sliding latches 145A, 147A to move radially outward underneath the projection 168A of the dome-shaped fastening member 161A. Rear angled surfaces 190A, 193A of the sliding latches 145A, 147A ride along the front surface of the projection 168A of the dome-shaped fastening member 161A, which is slightly angled toward the rear of the dome-shaped fastening member 161A, as the sliding latches 145A, 147A move radially outward. The sliding latches 145A, 147A become wedged beneath the projection 168A as the sliding latches 145A, 147A move radially outward.

FIG. 9E illustrates the completed mechanical connection between the piston head 134A and the dome-shaped fastening member 161A in which the sliding latches 145A, 147A have moved to maximum outwardly displaced positions within the dome-shaped fastening member 161A. In this configuration, the projection 168A of the dome-shaped fastening member 161A is effectively pinched between a rear member 137A of the piston head 134A and the sliding latches 145A, 147A, resulting in a secure engagement between the piston head 134A and the dome-shaped fastening member 161A. As a result of the secure engagement of the piston head 134A to the dome-shaped fastening member 161A, the amount of slippage of the piston head 134A relative to the dome-shaped fastening member 161A can be reduced (e.g., minimized) and thus precise pumping can be achieved.

After mechanically coupling the piston head 134A of the PD cycler 102 to the dome-shaped fastening member 161A of the cassette 112, a priming technique is carried out to remove air from the cassette 112 and from the various lines 126, 128, 130, 132 connected to the cassette 112. To prime the cassette 112 and the lines 126, 128, 130, 132, the piston 133A and inflatable members 142 are typically operated to pump dialysate from the heater bag 124 to the drain and from each of the dialysate bags 122 to the drain. Dialysate is also passed (e.g., by gravity) from the heater bag 124 to the patient line 130 to force any air trapped in the patient line out of a hydrophobic filter positioned at the distal end of the patient line 130.

After priming is complete, the patient line 130 is connected to the patient and the PD cycler 102 is operated to drain any spent dialysate that was left in the patient's peritoneal cavity from a previous treatment. To drain the spent dialysate from the patient's peritoneal cavity, the inflatable members 142 of the PD cycler 102 are configured to create an open fluid flow path between the patient line 130 and the port 187A (shown in FIG. 4) of the pump chamber 138A, and the piston 133A is retracted to draw spent dialysate from the peritoneal cavity of the patient into the pump chamber 138A via the patient line 130, as shown in FIG. 9F. Because the piston head 134A is mechanically connected to the dome-shaped fastening member 161A and the dome-shaped fastening member 161A is attached to the membrane 140 of the cassette 112, the retraction of the piston 133A causes the dome-shaped fastening member 161A and the portion of the membrane 140 attached to the dome-shaped fastening member 161A to move rearwardly. As a result, the volume of the pump chamber 138A is increased and spent dialysate is drawn into the pump chamber 138A from the peritoneal cavity of the patient. The spent dialysate travels from the patient line 130 through the pressure sensing chamber 163A and then enters the pump chamber 138A via the port 187A. The pressure sensor 151A is able to monitor the pressure in the pressure sensing chamber 163A, which is approximately equal to the pressure in the pump chamber 138A, during this process. If the patient line 130 is occluded or leaking, the pressure sensor 151A can be used to detect the occlusion or leak and cause an alarm. In response, the peripheral devices 200 can be activated in a manner to wake the patient and to direct the patient's attention towards the patient line 130, as will be described in greater detail below.

Referring to FIG. 9G, after drawing the dialysate into the pump chamber 138A from the peritoneal cavity of the patient, the inflatable members 142 are configured to create an open fluid flow path between the port 185A (shown in FIG. 4) of the pump chamber 138A and the drain line 132, and the dialysate is forced out of the pump chamber 138A to the drain by advancing the piston 133A and decreasing the volume of the pump chamber 138A. The piston 133A is typically advanced until the dome-shaped fastening member 161A contacts or nearly contacts the inner surface of the recessed region of the base 156 so that substantially all of the dialysate is forced out of the fluid pump chamber 138A via the port 185A. The pressure sensor 151A can be used to detect any leaks or occlusions in the drain line 132 and to trigger an alarm while activating the peripheral devices 200 to wake the patient and to direct the patient's attention to the drain line 132.

During the patient drain phase of the treatment, the pistons 133A, 133B are typically alternately operated such that the piston 133A is retracted to draw spent dialysate solution into the pump chamber 138A from the patient while the piston 133B is advanced to pump spent dialysate solution from the pump chamber 138B to the drain and vice versa.

To begin the patient fill phase, the inflatable members 142 are configured to create a clear fluid flow path between the pump chamber 138A and the heater bag line 128, and then the piston 133A is retracted, as shown in FIG. 15F, to draw warm dialysate from the heater bag 124 to the pump chamber 138A. The warm dialysate travels from the heater bag 124 through the heater bag line 128 and into the pump chamber via the port 185A. The pressure sensor 151A can be used detect any leaks or occlusions in the heater bag line 128 and to trigger an alarm while activating the peripheral devices 200 to wake the patient and to direct the patient's attention to the heater bag line 128.

The warm dialysate is then delivered to the peritoneal cavity of the patient via the patient line 130 by configuring the inflatable members 142 to create a clear fluid flow path between the pump chamber 138A and the patient line 130 and advancing the piston 133A, as shown in FIG. 9G. The warm dialysate exits the pump chamber 138A via the port 187A and travels through the pressure sensing chamber 163A to the patient line 130 before reaching the peritoneal cavity of the patient. The pressure sensor 151A is able to monitor the pressure in the pressure sensing chamber 163A, which is approximately equal to the pressure in the pump chamber 138A, during this process. The pressure sensor 151A can be used detect any leaks or occlusions in the patient line 130 and to trigger an alarm while activating the peripheral devices 200 to wake the patient and to direct the patient's attention to the patient line 130.

During the patient fill phase of the treatment, the pistons 133A, 133B are typically alternately operated such that the piston 133A is retracted to draw warm dialysate into the pump chamber 138A from the heater bag 124 while the piston 133B is advanced to pump warm dialysate from the pump chamber 138B to the patient and vice versa. When the desired volume of dialysate has been pumped to the patient, the cycler 102 transitions from the patient fill phase to a dwell phase during which the dialysate is allowed to sit within the peritoneal cavity of the patient for a long period of time. During the dwell period, toxins cross the peritoneum of the patient into the dialysate from the patient's blood. As the dialysate dwells within the patient, the PD cycler 102 prepares fresh dialysate for delivery to the patient in a subsequent cycle. In particular, the PD cycler 102 pumps fresh dialysate from one of the four full dialysate bags 122 into the heater bag 124 for heating. To do this, the pump of the PD cycler 102 is activated to cause the pistons 133A, 133B to reciprocate and certain inflatable members 142 of the PD cycler 102 are inflated to cause the dialysate to be drawn into the fluid pump chambers 138A, 138B of the cassette 112 from the selected dialysate bag 122 via its associated line 126. The dialysate is then pumped from the fluid pump chambers 138A, 138B to the heater bag 124 via the heater bag line 128. The pressure sensor 151A can be used to detect any leaks or occlusions in the lines 126, 128 and to trigger an alarm while activating the peripheral devices 200 to wake the patient and to direct the patient's attention to the lines 126, 128.

After the dialysate has dwelled within the patient for the desired period of time, the spent dialysate is pumped from the patient to the drain in the manner described above. The heated dialysate is then pumped from the heater bag 124 to the patient where it dwells for a desired period of time. These steps are repeated with the dialysate from two of the three remaining dialysate bags 122. The dialysate from the last dialysate bag 122 is typically delivered to the patient and left in the patient until the subsequent PD treatment.

After completion of the PD treatment, the pistons 133A, 133B are retracted in a manner to disconnect the piston heads 134A, 134B from the dome-shaped fastening members 161A, 161B of the cassette. The door 108 of the PD cycler is then opened and the cassette 112 is removed from the cassette compartment 114 and discarded.

As mentioned above, alarms, some of which may require intervention by the patient, may be triggered at the PD cycler 102. For example, an alarm may indicate a condition with the PD system 100 that the patient must resolve in order to continue the treatment. Since PD treatments are often carried out overnight while the patient sleeps, the room in which the patient is treated is typically dark. The dialysis machine touch screen 118 may also be darkened and/or unlit to allow for better sleeping conditions. The peripheral devices 200 can be used to wake the patient and in certain cases to illuminate the PD system 100 in a way to allow the patient to resolve the condition that caused the alarm.

One or more of the peripheral devices 200 in communication with (e.g., wirelessly connected to) the PD cycler 102 may be activated in response to a triggered alarm. The peripheral devices 200 can be activated to wake the patient and/or draw the patient's attention to a part of the PD system 100 requiring attention. Once the alarm has been resolved (e.g., once the conditions of the alarm are no longer met), the activated peripheral devices 200 can be de-activated.

In some examples, the light 202 is activated and illuminates some or all of the area surrounding the dialysis system 100 to allow the patient to respond to the alarm. For example, if the alarm requires information to be read on the display 118 of the PD cycler 102, the light 210 could illuminate the display. As another example, if the alarm indicates that a certain component of the dialysis system must be checked, one of the lights 208, 210 could illuminate that component. The light 202 could also flash in response to an alarm. In some examples, the light could flash in a way that wakes the patient. In some examples, the light could flash in the location that requires the patient's attention. In some examples, the light could flash in a pattern that indicates the type of alarm that has been activated. Using steady and/or flashing lights to wake the patient and/or direct the patient's attention may be particularly beneficial for hearing-impaired patients who may not respond well to audible alarms.

In some examples, the speaker 204 emits sound to wake the patient or plays a message to indicate to the patient the source of the alarm or does a combination of the two. The speaker 204 can also recite verbal instructions to the patient, aiding the patient in finding the source of and resolving the alarm condition. Alerting the patient and providing feedback from the speaker 204 can be particularly beneficial for patients who are visually-impaired. In some examples, the cellular telephone 206 receives and displays a message indicating the source of the alarm or sends a message notifying another party of the alarm or does a combination of the two.

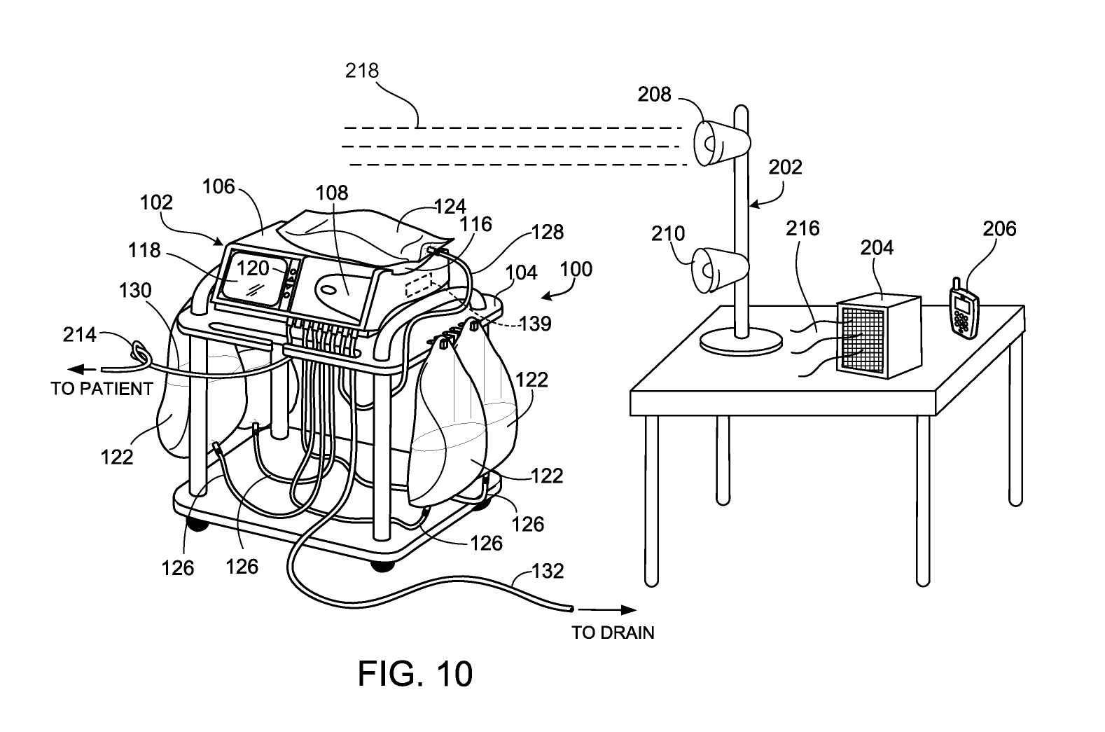

A few representative examples of the peripheral devices 200 and their activation in response to different alarms are included below. As shown in FIG. 10, a kink 214 has developed in the patient line 130 during treatment, causing the PD cycler 102 to trigger an alarm in response. The kink 214 inhibits the passing back and forth of dialysate between the patient and the cassette 112. As a result, the pressure sensors 151A, 151B, which are monitored by the control unit 139, detect a build-up of pressure. If the pressure readings remain elevated for a given length of time (e.g., 5 seconds), as is the case here, the PD cycler 102 triggers the alarm specified for a kinked patient line 130.

As a result of triggering the alarm, the PD cycler 102 sends activation data consistent with that alarm type over a communication link (e.g., wirelessly) to peripheral devices 200. In order to wake the patient, the PD cycler 102 activates the light 208 directed towards the patient and the speaker 204. The speaker 204 receives activation data directing it to play a sound and then emits a tone 216. The light 208 receives activation data directing it to flash and then emits a strobe 218 in the direction of the patient. Once the strobe and/or tone have woken the patient, the patient can press the control button 120 to indicate to the PD cycler 102 that the patient is awake. At that time the PD cycler 102 will communicate with the speaker 204, directing it to cease emitting the tone 216. The PD cycler 102 will also communicate with the light 208 directing it to quit emitting a strobe 218 and to instead shine a steady light, still in the direction of the patient. The continued use of the light 208 in the direction of the patient will indicate to the patient that the alarm was triggered by a condition in the direction of the patient (e.g., a kink 214 in the patient line 130). With the benefit of the illumination, the patient will be better able to find the kink 214. Once the kink 214 is corrected and dialysate is again able to pass through the patient line 130 at a desired rate, the control unit 139 in combination with the pressure sensors 151A, 151B will detect a drop in pressure, indicative of a resolution of the alarm condition. At that time the PD cycler 102 will communicate with the light 208, directing it to shut off. The room is returned to its pre-alarm state and treatment will resume.

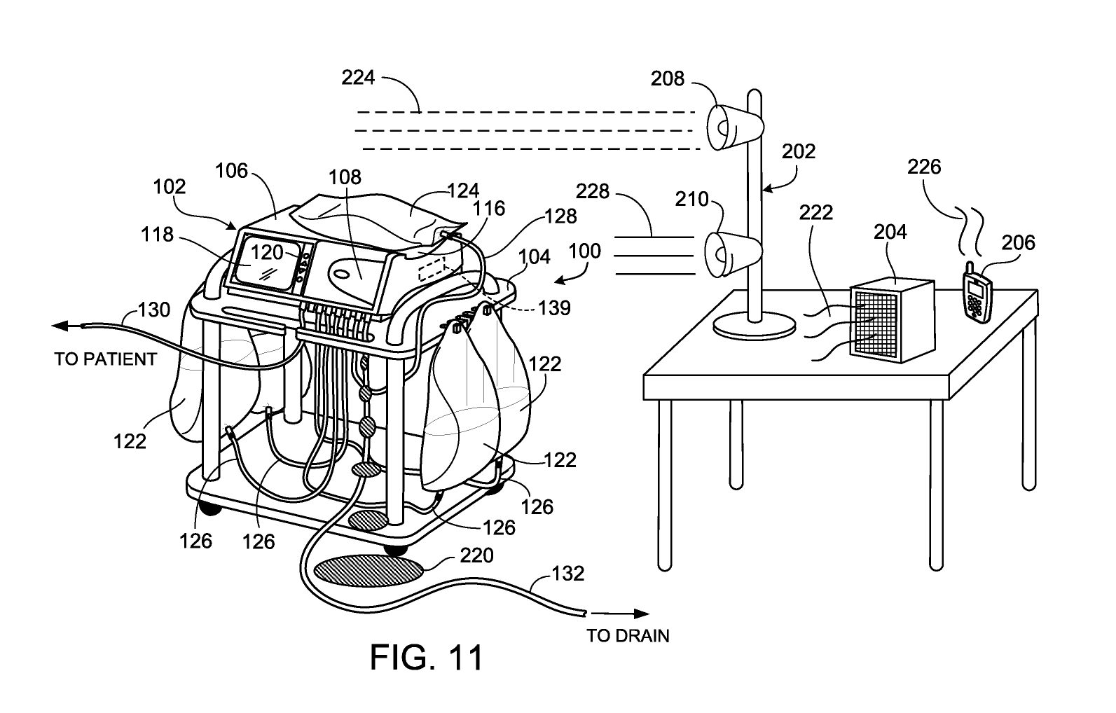

As shown in FIG. 11, a leak 220 has developed in one of the connectors 160 to which the lines 126, 128 are connected, causing the PD cycler 102 to trigger an alarm in response. A drop in pressure detected by pressure sensors 151A, 151B during the phase of treatment in which liquid is being delivered from one of the supply bags 122 or the heater bag 124 to the cassette 112 may be indicative of a leak 220 within the lines 126, 128 or the connectors 160 to which the lines 126, 128 are attached. Should pressure readings monitored by the control unit 139 remain below a threshold pressure for a given length of time (e.g., 5 seconds), the PD cycler 102 will trigger the corresponding alarm.

As a result of triggering the alarm, the PD cycler 102 sends activation data consistent with that alarm type over a communication link (e.g., wirelessly) to the peripheral devices 200. In order to wake the patient, the PD cycler 102 activates the light 208 directed towards the patient and speaker 204. The speaker 204 receives activation data directing it to play a sound and then emits a tone 222. The light 208 receives activation data directing it to flash and then emits a strobe 224 in the direction of the patient. The PD cycler 102 also activates the light 210, directing it to shine a steady light 228 on the front face of the PD cycler 102. The PD cycler 102 also activates the cellular telephone 206, notifying it to send an informational message 226 to a third party, such as a service center for the PD cycler 102, indicating that a leak 220 was detected.

Once the strobe and/or tone have woken the patient, the patient can press the control button 120 to indicate to the PD cycler 102 that the patient is awake. At that time the PD cycler 102 will communicate with the speaker 204, directing it to cease emitting the tone 222. The PD cycler 102 will also communicate with the light 208 directing it to quit emitting the strobe 224. The light 228 remains active, illuminating the front face of the PD cycler 102, and aiding the patient in finding the source of the leak 220. Once the patient locates the component that appears responsible for the leak 220, the patient may if possible replace that component. The patient, using control buttons 120, notifies the PD cycler 102 that the leak 220 has been resolved so that treatment will resume. If the control unit 139 in combination with the pressure sensors 151A, 151B detects a rise in pressure, confirming that the leak 220 has been resolved, the PD cycler 102 communicates with the light 210 directing it to turn off. If over the course of a treatment there are multiple instances of leak alarms that cannot be resolved, the control unit 139 will recognize the condition as a leak not able to be repaired by the patient. The PD cycler 102 will again wake the patient, and direct the patient to the possible location of the leak, as described above, but will also direct the cellular telephone 206 to send a more urgent message to a third party (e.g., a service center), notifying the third party of an unrecoverable leak condition. This allows for quick replacement of a malfunctioning PD cycler 102.

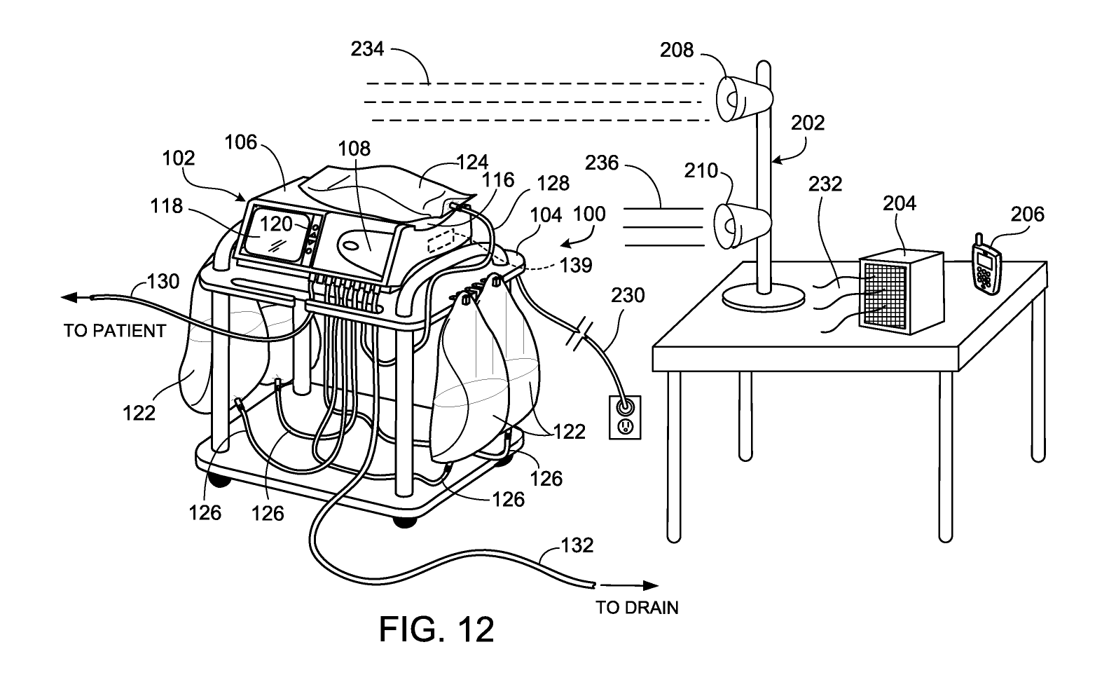

As shown in FIG. 12, a power loss 230 to the PD cycler 102 has occurred, causing the PD cycler 102 to trigger an alarm in response. Utilizing the backup power source, the PD cycler 102 sends activation data consistent with a power loss over a communication link (e.g., wirelessly) to the relevant peripheral devices 200. In order to wake the patient, the PD cycler 102 activates the lights 208, 210 directed towards the patient and PD cycler and the speaker 204. The speaker 204 receives activation data directing it to play a sound and then emits a tone 232. The light 208 receives activation data directing it to flash and then emits strobe a 234 in the direction of the patient. The light 210 receives activation data directing it to shine a steady light 236 on the PD cycler 102. Once the lights and/or tone have woken the patient, the patient can press the control button 120 to indicate to the PD cycler 102 that the patient is awake. At that time, the PD cycler 102 will communicate with the speaker 204, directing it to cease emitting the tone 216. The PD cycler 102 will also communicate with the light 208 directing it to quit emitting the strobe 234 and to instead shine a steady light, still in the direction of the patient. With the lights 208, 210 active and illuminating the PD cycler 102 and patient area, the patient will investigate the loss of power 230 and see if the cause is something that can be resolved (e.g. a loose electrical plug). If power is restored to the PD cycler 102, the PD cycler 102 will communicate with the lights 208, 210 directing them to shut off, and will cause treatment to resume. If the patient cannot resolve the power loss, the lights will remain active so that the patient can take any necessary actions, such as manually draining dialysate from his or her peritoneal cavity and possibly carrying out the remainder of the treatment manually.

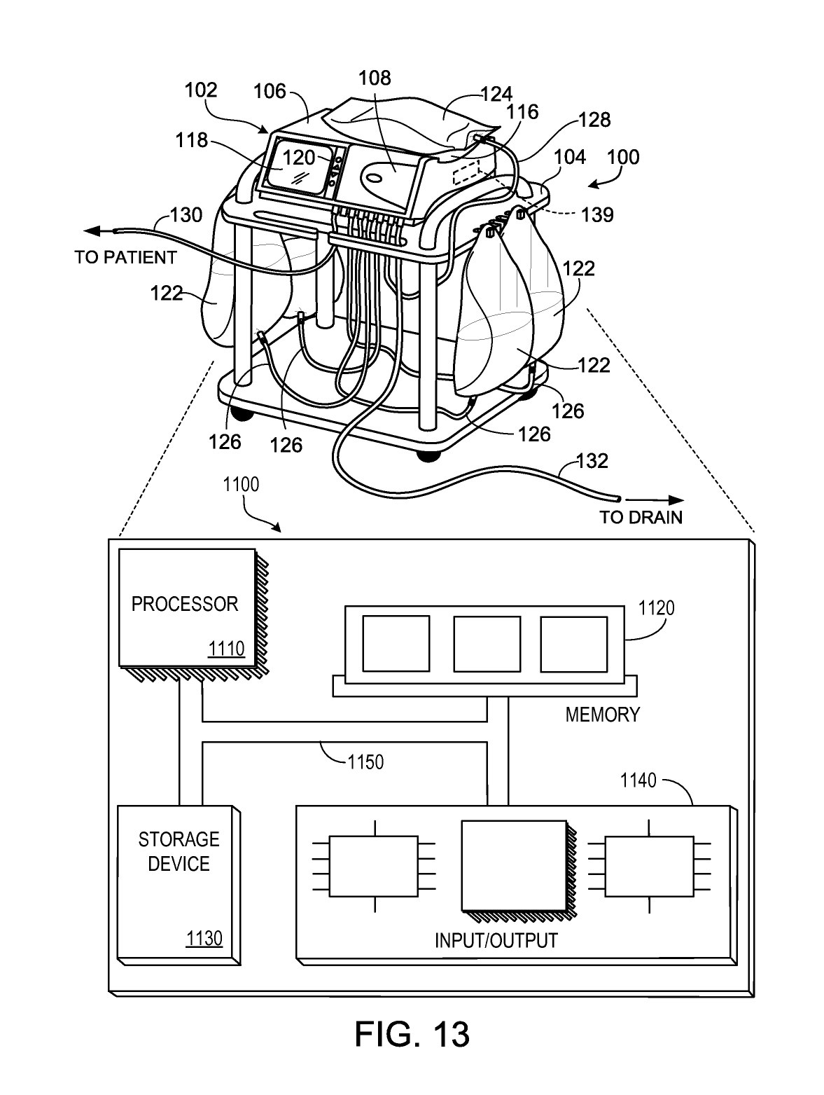

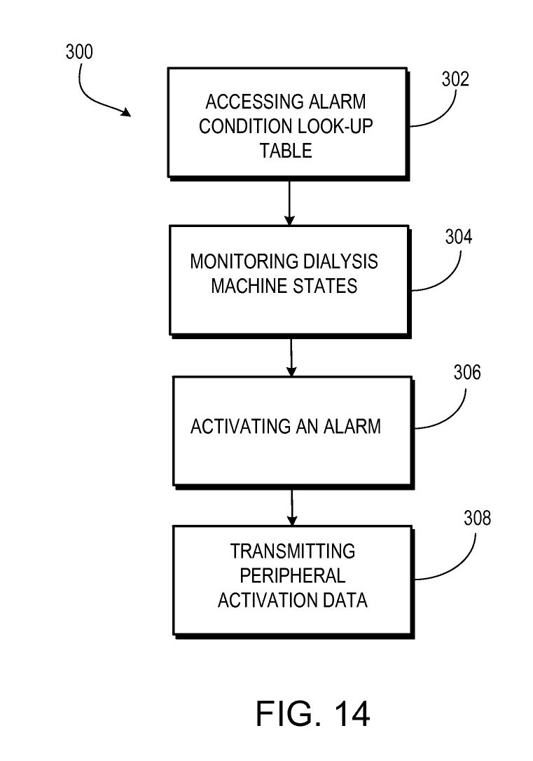

FIG. 13 is a block diagram of an example computer system 1100. For example, referring to FIG. 1, the control unit 139 could be an example of the system 1100 described here. The system 1100 includes a processor 1110, a memory 1120, a storage device 1130, and an input/output device 1140. Each of the components 1110, 1120, 1130, and 1140 can be interconnected, for example, using a system bus 1150. The processor 1110 is capable of processing instructions for execution within the system 1100. The processor 1110 can be a single-threaded processor, a multi-threaded processor, or a quantum computer. The processor 1110 is capable of processing instructions stored in the memory 1120 or on the storage device 1130. The processor 1110 may execute operations such as accessing a look-up table of alarm conditions and determining when an alarm criterion is satisfied (FIG. 14). The memory 1120 stores information within the system 1100. In some implementations, the memory 1120 is a computer-readable medium. The memory 1120 can, for example, be a volatile memory unit or a non-volatile memory unit.

The storage device 1130 is capable of providing mass storage for the system 1100. In some implementations, the storage device 1130 is a non-transitory computer-readable medium. The storage device 1130 can include, for example, a hard disk device, an optical disk device, a solid-date drive, a flash drive, magnetic tape, or some other large capacity storage device. The storage device 1130 may alternatively be a cloud storage device, e.g., a logical storage device including multiple physical storage devices distributed on a network and accessed using a network.

The input/output device 1140 provides input/output operations for the system 1100. In some implementations, the input/output device 1140 includes one or more of network interface devices (e.g., an Ethernet card), a serial communication device (e.g., an RS-232 10 port), and/or a wireless interface device (e.g., an 802.11 card, a 3G wireless modem, or a 4G wireless modem). A network interface device allows the system 1100 to communicate, for example, transmit and receive data such peripheral activation data, shown in FIG. 14. In some implementations, the input/output device includes driver devices configured to receive input data and send output data to other input/output devices, e.g., keyboard, printer and display devices 118. In some implementations, mobile computing devices, mobile communication devices, and other devices are used.

Although an example processing system has been described in FIG. 13, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification, such as software for configuring and accessing a look-up table of alarm conditions (FIG. 14), can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, a composition of matter effecting a machine readable propagated signal, or a combination of one or more of them.

The term “computer system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM and DVD-ROM disks. The processor and the memory can be supplemented by, or incorporated in, special purpose logic circuitry. The components of the system can be interconnected by any form or medium of digital data communication, e.g., a communication network. Examples of communication networks include a local area network (“LAN”) and a wide area network (“WAN”), e.g., the Internet.

In some implementations, the dialysis system 100 can be configured with a look-up table that associates alarm condition criteria and references to data that can be used to activate a combination of peripheral devices. FIGS. 10, 11, and 12 illustrate example peripheral activations as specified by an example look-up table configuration. In some examples, the look-up table may be configured differently to cause different alarms to trigger in response to different criteria, and to activate different peripheral devices in response to those alarms. In some examples, the look-up table may reside in memory 1120 or a storage device 1130 (FIG. 13) or a combination of the two. In some examples, the look-up table may be configured and accessed by instructions running on a processor 1110. A sample look-up table is shown below.

| Alarm Condition | Criteria | Peripheral Activation Data |
|---|---|---|
| Kink in patient line | i) threshold pressure in | i) flash light on patient/ |
| | patient line | steady light on patient |
| | ii) threshold time at which | ii) play sound from speaker |
| | pressure is above threshold | |
| | pressure | |
| Leak in dialysate bag line or | i) threshold pressure in | i) flash light on patient |
| heater bag line | dialysate bag line or heater | ii) steady light on PD cycler |
| | bag line | iii) play sound from speaker |
| | ii) threshold time at which | iv) send message from |
| | pressure is below threshold | cellular telephone |
| | pressure | |
| PD Cycler has lost main | i) electrical signal | i) flash light on patient/ |
| power | undetectable on main power | steady light on patient |
| | line | ii) steady light on PD cycler |
| | ii) threshold time at which | iii) play sound from speaker |
| | electrical signal has been | |
| | undetectable | |

FIG. 14 is a flowchart showing an example process 300. The process 300 can be carried out, for example, by the PD cycler 102 shown in FIG. 1.

As shown in FIG. 14, a look-up table of one or more alarm conditions associated with the PD cycler 102 is accessed 302. At least some of the alarm conditions include one or more alarm criteria, and at least some of the alarm conditions include peripheral activation data. In some examples, the look-up table access 302 occurs continuously. In some examples, the look-up table access 302 occurs once initially, and additional accesses occur in response to look-up table changes.

The states of the PD cycler 102 are continuously monitored 304. In some examples, each state is associated with at least one of the alarm criteria. If the alarm criteria of an alarm condition are satisfied, then the alarm corresponding to that alarm condition is activated 306. In some examples, the alarm condition is indicative of a patient-correctable condition, e.g. the malfunction of a component of the dialysis system able to be replaced by the patient. In some examples, the alarm condition is indicative of a malfunction that cannot be corrected by the patient.

The PD cycler 102 transmits 308 peripheral activation data to a specified peripheral device among the peripheral devices 200. In some examples, the peripheral activation data may indicate a one-time action (e.g., activate light). In some examples, the peripheral activation data may indicate an ongoing action (e.g., flash light for a period of time, or flash light until the alarm condition is resolved). In some examples, the peripheral is electrically isolated from the PD cycler. In some examples, the peripheral activation data is transmitted wirelessly. In some examples, instructions for remedying the malfunction are displayed on the touch screen 118. In some examples, peripheral devices are activated and provide feedback drawing the patient's attention to a replaceable part of the PD system 100 (e.g., a disposable component). In some examples, the feedback involves illuminating a specific part of the PD system 100.

### Alternative Embodiments

While the alarm conditions have been described as a kink in the patient line 130, a leak in the dialysate bag line 126 or heater line 128, or a loss of power to the PD cycler 102, alarms can trigger from other various conditions. It will be understood that the peripheral devices 200 that are activated and the ways in which these peripheral devices 200 are activated will vary from one type of alarm condition to another.

While the alarm conditions have been described as being detected based on readings of the pressure sensors 151A, 151B or a loss of power, other readings can trigger an alarm. For example, an alarm may trigger based on the position of pistons 133A, 133B read from the encoders. One such alarm is an empty heater bag alarm. If the control unit 139 determines, based on the positions of the pistons 133A, 133B at certain reference pressures, that the heater bag 124 is empty, the control unit 139 can activate the peripheral devices 200 to wake the patient and to draw the patient's attention to the heater bag 124 using any of the various techniques described herein.

While the peripheral devices 200 have been described as being activated in response to alarm conditions during the treatment phase, the peripheral devices 200 can also be activated in response to an alarm occurring during the setup or priming phase. Different alarms conditions may be detected during the setup and priming phases, and different peripheral devices 200 may be activated in response to those alarms. For example, while the peripheral devices 200 are typically activated in a manner to wake a patient in response to alarms that occur during treatment, the patient will typically be awake during the setup and priming phases, rendering activation of those particular peripheral devices 200 unnecessary. Thus, in certain cases, the peripheral devices 200 will only be activated in a manner to draw the patient's attention to a possible problem in response to alarms that occur during the setup or priming phase.

While the peripheral devices 200 have been described as a speaker 204, a cellular telephone 206, and a light 202, the peripheral devices 200 could alternatively or additionally include other types of devices, such as a buzzer that generates a vibration to wake a patient, identifies a location at which attention is needed, indicates a type of alarm activated, and so on. Any combination of peripheral devices could be used.

While the peripheral devices 200 have been describes as wirelessly communicating 212 with the PD cycler 102, the peripheral devices can alternatively communicate with the PD cycler 102 over a hard wire. Additionally, the peripheral devices 200 can be integrated into the PD cycler 102 itself in certain cases. While the light elements 208, 210 have been described as statically pointing in a certain direction, in some implementations, the lights 208, 210 are motorized. In such implementations, signals sent to the lights 208, 210 can manipulate the motorized controls and change the direction in which the lights 208, 210 point. As a result, a small number of lights (e.g., a single light) can be used to direct the patient's attention to multiple different parts of the system in response to different alarm conditions.

While the light 202 has been described as a table-mounted light, the light could alternatively be a room light (e.g., ceiling light) with light heads directed to different areas of the room and also capable of illuminating the entire room for certain alarms.

While the light 202 has been described as using lighting elements pointed in different directions 208, 210 to indicate the source of an alarm condition, the light 202 could alternatively use other feedback methods to indicate the type of problem (e.g., activating differently colored lights for different alarms, utilizing different strobe frequencies).

While the speaker 204 has been described as emitting a tone, the speaker could alternatively playback a message indicating the source of the alarm.

While the cellular telephone 206 has been described as sending messages to a third party (e.g., a service center) in response to certain types of alarms, the cellular telephone 206 could alternatively send a message in response to all alarms notifying the recipient that certain alarm conditions were detected. In some implementations, a mobile device other than a cellular telephone (e.g., a mobile device such as a tablet computer) could be used in place of the cellular telephone 206 and provide substantially the same functionality as a cellular telephone.

In some implementations, a peripheral device providing touch feedback (e.g., tactile feedback or haptic feedback) could be used. For example, a peripheral device could provide touch feedback to a user who is visually or aurally impaired.

While the dialysate has been described as being pumped into the heater bag 124 from a single dialysate bag 122, dialysate can alternatively be pumped into the heater bag 124 from multiple dialysate bags 122. Such a technique may be advantageous, for example, where the dialysates in the bags 122 have different concentrations (e.g., different dextrose concentrations) and a desired concentration for treatment is intermediate to the concentrations of the dialysate in two or more of the bags 122.

While the piston heads 134A, 134B have been described as including spring-loaded latch mechanisms with sliding latches 145A, 145B that can be move radially inward and outward to allow those piston heads 134A, 134B to be mechanically connected to the dome-shaped fastening members 161A, 161B of the cassette 112, piston heads of simpler construction that include no such sliding latches can alternatively be used in some cases. In some implementations, for example, each of the piston heads is a unitary structure that includes a peripheral flange that can be engaged with an annular projection of a dome-shaped member of a cassette in order to mechanically connect the piston head to the cassette and enable a fluid pumping process of the type described above to be carried out. In such implementations, the rear surface of the flange can be arranged at an angle of about 45 degrees to about 75 degrees (e.g., about 60 degrees) relative to the longitudinal axis of the piston to facilitate insertion of the piston head into the dome-shaped member. The peripheral flange of the piston head and/or the flange of the dome-shaped member can elastically deform as the piston head is advanced into the dome-shaped member. Examples of this type of piston head and dome-shaped member as well as other suitable types of piston heads and dome-shaped members are described in U.S. Patent Application Publication No. 2012/0271226, which is incorporated by reference herein.

While the piston heads and dome-shaped members of the cassette have been described above as being mechanically coupled to one another, other coupling techniques can be used. In some implementations, for example, the cassette includes a membrane that overlies the entire area of the pump chambers and that is driven by dome-shaped piston heads that generally conform to the recessed regions of the rigid base of the cassette. In such implementations, the cassette interface typically includes annular openings surrounding the piston heads via which vacuum can be applied from a vacuum source (e.g., a vacuum pump or a negatively pressurized vacuum chamber) to the cassette membrane to hold the portions of the cassette membrane overlying the pump chambers in contact with the piston heads. Examples of such systems can be found in U.S. Patent Application Publication No. 2007/0112297, which is incorporated by reference herein. Because the outer diameter of much of the piston heads is smaller than the maximum inner diameter of the recessed regions of the rigid base of the cassette, throughout most of the piston stroke, the membrane will include annular portions that surround the piston head and overlie the pump chambers. In much the same way as discussed above, these annular portions will tend to bulge outward and inward as fluid is pumped out of and drawn into the pump chambers by advancing and retracting the pistons, respectively. Thus, the volume of fluid that is pumped out of and drawn into the pump chambers can be determined with greater accuracy by using a correction factor that accounts for the bulging of the annular portions of the membrane. The correction factors can be selected and applied using the processes discussed above.

While the cassette interface 110 of the PD cycler 102 has been described as including locating pins 148 that help to ensure that the dome-shaped members of the cassette are aligned with the pistons 133A, 133B when the cassette is positioned in the cassette compartment 114, other structures or techniques can be used to ensure this alignment. In some implementations, for example, the cassette is placed against the door of the PD cycler with the hollow projections of the cassette disposed in recesses of the PD cycler's door, and the cassette is held in this position by retainer clips attached to the door. Upon closing the door, the pistons of the PD cycler align with the dome-shaped members of the cassette.

While the door 108 of each of the PD cyclers above has been described as including an inflatable pad that, when inflated, can press the cassette against the cassette interface, the inflatable pad can alternatively be positioned behind the cassette interface such that the cassette interface can be moved toward the door 108 to compress the cassette therebetween. Similarly, as an alternative to an inflatable pad, any of various mechanisms that can be operated to move a surface of the door 108 toward the cassette interface or vice versa can be used.

While the door 108 of the PD cyclers described above are shown as being positioned on a front face of the PD cyclers, the doors can alternatively be positioned at various other locations on the PD cyclers. For example, the doors could be positioned on a top face of the PD cycler such that the cassette is slid into the cassette compartment in a substantially horizontal orientation instead of a substantially vertical orientation. In some implementations, the door and the cassette interface of the PD cycler are positioned at an angle of about 10 to about 35 degrees to vertical when the PD cycler is rested on a horizontal surface. It has been found that this configuration makes it easier for the user to load the cassette into the cassette compartment. While the cassettes discussed above have two pump chambers, the cassettes can alternatively have more or fewer than two pump chambers.

While each of the pump chambers of the cassettes described above has been described as including multiple ports, in certain implementations, the pump chambers include a single port that is used as both an inlet and an outlet. In such implementations, the inflatable valve members of the PD cycler that act on the valve portions of the cassettes would be activated and deactivated in a different sequence to allow fluid to be drawn into the pump chamber from a desired location and then to be forced out of the pump chamber to a desired location.

While certain PD cyclers above have been described as including a touch screen and associated buttons, the PD cyclers can alternatively or additionally include other types of screens and user data entry systems. In certain implementations, for example, the cycler includes a display screen with buttons (e.g., feather touch buttons) arranged on the console adjacent the display screen. Certain buttons can be arranged to be aligned with operational options displayed on the screen during use such that the user can select a desired operational option by pressing the button aligned with that operational option. Additional buttons in the form of arrow buttons can also be provided to allow the user to navigate through the various display screens and/or the various items displayed on a particular screen. Other buttons can be in the form of a numerical keypad to allow the user to input numerical values in order, for example, to input operational parameters. A select or enter button can also be provided to allow the user to select an operational option to which the user navigated by using the arrow keys and/or to allow the user to enter values that the user inputted using the numerical keypad.

While PD systems have been described, the methods described herein can be used in any of various other types of medical fluid pumping systems. Other examples of medical fluid pumping systems with which the methods described herein can be used include hemodialysis systems, blood perfusion systems, and intravenous infusion systems.

Similarly, while many of the systems above have been described as being used to pump dialysate, other types of dialysis fluids can be pumped through the cassettes. As an example, in the case of cassettes used with hemodialysis machines, blood can be pumped through the cassettes. In addition, priming solutions, such as saline, can similarly be pumped through cassettes using the various different systems and techniques described above. Similarly, as an alternative to dialysis fluids, any of various other types of medical fluids can be pumped through the above-described cassettes depending on the type of medical fluid pumping machines with which the cassettes are used.

Other embodiments are within the scope of the following claims.

1. A method comprising:
determining that one or more alarm criteria of a first alarm condition of a dialysis machine is satisfied; and
in response to determining that the one or more alarm criteria of the first alarm condition is satisfied, activating an alarm corresponding to the first alarm condition and activating a peripheral device.

2. The method of claim 1, wherein the peripheral device is activated by transmitting to the peripheral device peripheral activation data associated with the first alarm condition.

3. The method of claim 2, wherein the peripheral activation data associated with the first alarm condition is wirelessly transmitted to the peripheral device.

4. The method of claim 1, wherein determining that the one or more alarm criteria of the first alarm condition is satisfied comprises monitoring data associated with the dialysis machine and comparing the data to the one or more alarm criteria of the first alarm condition.

5. The method of claim 4, wherein the data associated with the dialysis machine comprises pressure data.

6. The method of claim 1, wherein determining that the one or more alarm criteria of the first alarm condition is satisfied comprises accessing a look-up table that comprises the first alarm condition and the one or more alarm criteria and peripheral activation data associated with the first alarm condition.

7. (canceled)

8. The method of claim 1, wherein the method comprises activating a plurality of peripheral devices in response to determining that the one or more alarm criteria of the first alarm condition is satisfied, at least one of the peripheral devices waking a patient upon being activated and at least one of the peripheral devices drawing the patient's attention to a particular part of the system upon being activated.

9. (canceled)

10. The method of claim 1, wherein activating the peripheral device causes the peripheral device to transmit data to a remote location.

11. (canceled)

12. The method of claim 1, further comprising, in response to determining that one or more alarm criteria of a second alarm condition is satisfied, activating an alarm corresponding to the second alarm condition and activating a peripheral device.

13. The method of claim 12, wherein the peripheral device activated in response to determining that the one or more alarm criteria of the second alarm condition is satisfied differs from the peripheral device activated in response to determining that the one or more alarm criteria of the first alarm condition is satisfied.

14. The method of claim 12, wherein activating the peripheral device in response to determining that the one or more alarm criteria of the first alarm condition is satisfied causes a first part of the dialysis machine to be illuminated, and activating the peripheral device in response to determining that the one or more alarm criteria of the second alarm condition is satisfied causes a second part of the dialysis machine to be illuminated.

15. The method of claim 12, wherein, in response to determining that the one or more alarm criteria of the first alarm condition is satisfied, a plurality of first peripheral devices are activated, at least one of the first peripheral devices waking a patient upon being activated and at least one of the first peripheral devices drawing the patient's attention to a particular part of the system upon being activated.

16-18. (canceled)

19. The method of claim 1, wherein the first alarm condition is indicative of a condition of a disposable component of the dialysis system.

20. The method of claim 19, wherein the first alarm condition is indicative of a malfunction of the disposable component.

21. (canceled)

22. The method of claim 19, wherein activating the peripheral device causes the peripheral device to provide feedback drawing a patient's attention to the disposable component.

23. (canceled)

24. A computer readable storage device storing instructions that, when executed, cause a computer system to perform operations comprising:
accessing a look-up table of a plurality of alarm conditions associated with a dialysis machine, each of the alarm conditions being associated with one or more alarm criteria and one or more peripheral activation data;
monitoring one or more states of the dialysis machine, each state being associated with the one or more alarm criteria of at least one of the alarm conditions;
in response to determining that the one or more alarm criteria associated with a first alarm condition is satisfied, activating an alarm corresponding to the first alarm condition; and
transmitting, to a peripheral device, peripheral activation data associated with the first alarm condition.

25. A dialysis system comprising
a monitoring circuit configured to identify an alarm condition of the dialysis machine, the alarm condition identified based on one or more criteria associated with the alarm condition, and the alarm condition associated with peripheral activation data; and
an alarm circuit configured to transmit the peripheral activation data to a peripheral device based on the identified alarm condition, the peripheral activation data comprising data to cause the peripheral device to operate in a mode of the peripheral device relevant to the alarm condition.

26. The dialysis system of claim 25, wherein the alarm condition is an indication that a component of the dialysis system may be malfunctioning, and the mode of the peripheral device comprises providing feedback drawing attention to the component of the dialysis system.

27. The dialysis system of claim 26, wherein providing feedback drawing attention to the component of the dialysis system comprises illuminating the component of the dialysis system.

28. The dialysis system of claim 25, further comprising a plurality of peripheral devices that are configured to receive peripheral activation data from the alarm circuit.

29. The dialysis system of claim 28, wherein at least one of the peripheral devices is configured to wake a patient upon receiving the peripheral activation data and at least one of the peripheral devices is configured to draw the patient's attention to a particular part of the system upon receiving the peripheral activation data.

30. The dialysis system of claim 29, wherein the at least one peripheral device that is configured to wake the patient comprises a speaker and/or a light directed to the patient, and the at least one peripheral device that is configured to draw the patient's attention to a particular part of the system comprises a light directed to the particular part of the system.

31. The dialysis system of claim 28, wherein at least one of the peripheral devices is configured to transmit information to a remote location upon receiving the peripheral activation data.

32-34. (canceled)

35. The dialysis system of claim 25, wherein the peripheral device is in wireless communication with the dialysis machine.

36. (canceled)

37. The dialysis system of claim 25, wherein the alarm condition is indicative of a condition of a disposable component of the dialysis system.

38. The dialysis system of claim 37, wherein the alarm condition is indicative of a malfunction of the disposable component.

39. (canceled)

40. The dialysis system of claim 37, wherein the mode of the peripheral device comprises providing feedback drawing attention to the disposable component.

41. (canceled)

42. The dialysis system of claim 37, wherein the disposable component comprises a tube.

43. The dialysis system of claim 42, wherein the one or more criteria associated with the alarm condition comprises a pressure that exceeds a threshold pressure.

44. (canceled)

45. The dialysis system of claim 25, comprising a look up table of alarm condition entries, each alarm condition entry being associated with i) one or more criteria and ii) peripheral activation data.

46. (canceled)
